(19) United States
(12) Patent Application Publication
(10) Pub. No.: US 2004/0260097 A1
(43) Pub. Date: Dec. 23, 2004

(54) Insecticidal and acaricidal 3-substituted pyrazoles

(76) Inventors: Joseph A. Furch, Lawrenceville, NJ (US); David Kuhn, Newtown, PA (US); Stephen S. Szucs, Lawrenceville, NJ (US); Kun-jian Gu, Lawrenceville, NJ (US); Wolfgang von Deyn, Neustadt (DE)

Correspondence Address: KEIL & WEINKAUF, 1350 CONNECTICUT AVENUE, N.W., WASHINGTON, DC, 20036, US

(21) Appl. No.: 10/490,369

(22) PCT Filed: Sep. 25, 2002
(86) PCT No.: PCT/EP02/10719

(30) Foreign Application Priority Data
Sep. 25, 2001 (US) 60324633

(51) Int. Cl. C 07D 4 3/02

(52) U.S. Cl. 546/275.4; 546/276.1

(57) ABSTRACT

Compounds of formula (I), wherein the variables and the index have the following meanings: R¹ H, halogen, alkyl, haloalkyl, alkenyl, haloalkenyl, alkylthio, alkoxyalkyl, alkylthioalkyl, or optionally substituted phenyl; R² H, halogen, alkyl, haloalkyl, alkenyl, haloalkenyl or optionally substituted phenyl; A H, OH, CN, NO₂, halogen, SCN, alkoxy, haloalkoxy, alkenyloxy, alkylthio, haloalkylthio, alkylsulfinyl, alkylsulfonyl, aminothiocarbonyl, hydroxycarbonyl, alkoxycarbonyl, or aminocarbonyl; B H, OH, NH₂, CN, NO₂, halogen, optionally substituted alkyl, optionally substituted alkoxy, optionally substituted alkenyl, alkenyloxy, alkylthio, haloalkylthio, alkoxythiocarbonylthio, alkoxycarbonylalkoxy, alkoxycarbonylalkylthio, alkylsulfinyl, alkylsulfonyl, aminothiocarbonyl, NR³R⁴, N═CHOR⁵, or N═CHNR⁵; R³, R⁴ H, alkyl, alkoxycarbonylalkyl, [(alkoxycarbonyl)(alkenyl)]alkyl, alkoxycarbonylalkenyl, alkylcarbonyl, cycloalkylcarbonyl, alkylaminocarbonyl, diaminocarbonyl, alkoxycarbonyl, alkoxyaminosulfonyl, or di(alkoxy)aminosulfonyl; R⁵ alkyl, haloalkyl, or phenylalkyl; Q H, NO₂, halogen, haloalkyl, alkylamino, dialkylamino, alkoxy, haloalkoxy, or alkenyloxy; X H, halogen, haloalkyl, alkoxy or haloalkoxy; Y H, halogen, haloalkyl, alkoxy or haloalkoxy; Z H, halogen, haloalkyl, alkoxy or haloalkoxy; M N or CR⁶; R⁶ H, NO₂, halogen or haloalkyl; n 0, 1, 2, 3, or 4, with the proviso that, when R¹ is hydrogen, n is not zero, processes for the preparation of compounds of formula (I), compositions containing them and their use for the control of insect and acarid pests and for the protection of plants from those pests as well as their use for treating, controlling, preventing and protecting warm-blooded animals and humans against infestation and infection by arachnids and arthropod endo-and ectoparasites.

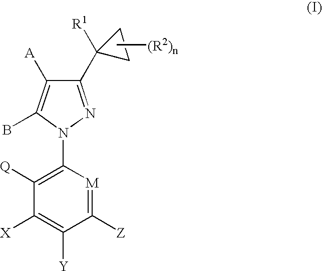

The present invention provides compounds of formula I

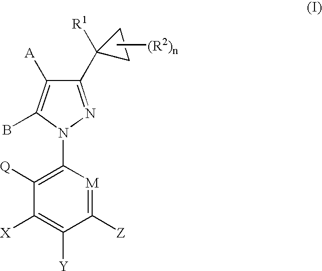

wherein the variables and the index have the following meanings:

R¹ hydrogen, halogen, C₁-C₆-alkyl, C₁-C₆-haloalkyl, C₂-C₆-alkenyl, C₂-C₆-haloalkenyl, C₁-C₆-alkylthio, C₁-C₆-alkoxy-C₁-C₄-alkyl, Cₗ-C₆-alkylthio-C₁-C₄-alkyl, or phenyl which is unsubstituted or substituted with 1 to 3 groups R^{a};

R^{a} halogen, nitro, cyano, C₁-C₆-alkyl, C₁-C₆-haloalkyl, C₁-C₆-alkylthio, C₁-C₆-haloalkylthio, C₁-C₆-alkoxy or C₁-C₆-haloalkoxy;

R² hydrogen, halogen, C₁-C₆-alkyl, C₁-C₆-haloalkyl, C₂-C₆-alkenyl, C₂-C₆-haloalkenyl or phenyl which is unsubstituted or substituted with 1 to 3 groups R^{a};

A hydrogen, hydroxy, cyano, nitro, halogen, rhodano, C₁-C₆-alkoxy, C₁-C₆-haloalkoxy, C₂-C₆-alkenyloxy, C₁-C₆-alkylthio, C₁-C₆-haloalkylthio, C₁-C₆-alkylsulfinyl, C₁-C₆-alkylsulfonyl, aminothiocarbonyl, hydroxycarbonyl, C₁-C₆-alkoxycarbonyl, aminocarbonyl;

B hydrogen, hydroxy, amino, cyano, nitro, halogen, C₁-C₆-alkyl, unsubstituted or substituted by one to three groups selected from halogen and cyano; C₁-C₆-alkoxy, unsubstituted or substituted by one to three groups selected from halogen, cyano, C₂-C₄-alkenyl, and C₁-C₆-alkoxycarbonyl-C₂-C₄-alkenyl; C₂-C₆-alkenyl, unsubstituted or substituted by one to three groups selected from halogen and cyano; C₂-C₆-alkenyloxy, C₁-C₆-alkylthio, C₁-C₆-haloalkylthio, Cₗ-C₆-alkoxythiocarbonylthio, Cₗ-C₆-alkoxycarbonyl-C₁-C₄-alkoxy, C₁-C₆-alkoxycarbonyl-C₁-C₄-alkylthio, C₁-C₆-alkylsulfinyl, C₁-C₆-alkylsulfonyl, aminothiocarbonyl, NR³R⁴, N═CHOR⁵, or N═CHNR⁵;

R³,R⁴ each independently hydrogen, C₁-C₆-alkyl, C₁-C₆-alkoxycarbonyl-C₁-C₄-alkyl, [(C₁-C₆-alkoxycarbonyl)(C₂-C₄-alkenyl)]C₁-C₄-alkyl, C₁-C₆-alkoxycarbonyl-C₂-C₄-alkenyl, Cₗ-C₆-alkyl-carbonyl, C₃-C₇-cycloalkyl-carbonyl, C₁-C₆-alkyl-aminocarbonyl, di-(C₁-C₆-alkyl)aminocarbonyl, C₁-C₆-alkoxycarbonyl, C₁-C₆-alkoxy-aminosulfonyl, or di-(C₁-C₆-alkoxy)aminosulfonyl;

R⁵ C₁-C₆-alkyl, C₁-C₆-haloalkyl, or phenyl-C₁-C₄-alkyl;

Q hydrogen, nitro, halogen, C₁-C₄-haloalkyl, C₁-C₆-alkylamino, di-(C₁-C₆)-alkylamino, C₁-C₆-alkoxy, C₁-C₆-haloalkoxy, C₂-C₆-alkenyloxy;

X hydrogen, halogen, C₁-C₆-haloalkyl, C₁-C₆-alkoxy or C₁-C₆-haloalkoxy;

Y hydrogen, halogen, C₁-C₆-haloalkyl, C₁-C₆-alkoxy or C₁-C₆-haloalkoxy;

Z hydrogen, halogen, C₁-C₆-haloalkyl, C₁-C₆-alkoxy or Cₗ-C₆-haloalkoxy;

M N or CR⁶;

R⁶ hydrogen, nitro, halogen or C₁-C₄-haloalkyl;

n 0, 1, 2, 3, or 4,

with the proviso that, when R¹ is hydrogen, n is not zero.

Furthermore, the present invention relates to processes for the preparation of compounds of formula I, compositions containing them and their use for the control of insect and acarid pests and for the protection of plants from those pests as well as their use for treating, controlling, preventing and protecting warm-blooded animals and humans against infestation and infection by arachnids and arthropod endo- and ectoparasites.

Pyrazoles such as those described in WO 98/45274 or U.S. Pat. No. 5,232,940 are known to demonstrate insecticidal and parasiticidal activity.

WO 98/24767 discloses parasiticidally active pyrazoles carrying a cyclopropyl group in the 4-position.

In EP-A 200 872, pesticidal pyrazoles are described that carry a NO₂-group in the 4-position and may carry a C₃-C₇-cycloalkyl group in the 3-position.

However, the pesticidal activity of the compounds known from the above literature in many cases is unsatisfactory.

It is therefore an object of the present invention to provide further compounds having improved insecticidal and acaricidal activity.

It is also an object of the present invention to provide compounds having improved parasiticidal activity.

We have found that these objects are achieved by pyrazole derivatives of formula I. Furthermore, we have found processes for preparing the compounds of formula I and the use of the compounds I and compositions comprising them for use for the control of insects and arachnids and for the protection of growing and harvested crops and wooden structures from damage caused by insect and acarid attack and infestation, as well as the use of compounds of formula I for treating, controlling, preventing and protecting warm-blooded animals and humans against infestation and infection by arachnids and arthropod endo- and ectoparasites.

The pyrazole moiety of the compounds described in WO 98/45274 or U.S. Pat. No. 5,232,940 is not substituted by a cycloalkyl group.

Contrary to the parasiticidal compounds disclosed in WO 98/24767, the inventive compounds of formula I carry a cyclopropyl group in the 3-position of the pyrazole moiety.

The compounds of formula I differ from the compounds known from EP-A 200 872 in that the pyrazole moiety is substituted by cyclopropyl.

Depending on the substitution pattern, the compounds of formula I can contain one or more chiral centers, in which case they are present as enantiomer or diastereomer mixtures. Subject matter of the invention are the pure enantiomers or diastereomers as well as their mixtures.

In the definitions of the symbols given in the above formulae, and throughout the description and claims, collective terms are used which generally represent the following substituents:

Halogen: fluoro, chloro, bromo and iodo;

Alkyl: saturated, straight-chain or branched hydrocarbon radicals having 1 to 4 or 6 carbon atoms, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;

Haloalkyl: straight-chain or branched alkyl groups having 1 to 4 or 6 carbon atoms (as mentioned above), where some or all of the hydrogen atoms in these groups may be replaced by halogen atoms as mentioned above, for example C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl and pentafluoroethyl;

Alkenyl: unsaturated, straight-chain or branched hydrocarbon radicals having 2 to 6 carbon atoms and a double bond in any position, such as ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl; 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

Haloalkenyl: unsaturated, straight-chain or branched hydrocarbon radicals having 2 to 6 carbon atoms and a double bond in any position (as mentioned above), where some or all of the hydrogen atoms in these groups may be replaced by halogen atoms as mentioned above, in particular by fluoro, chloro and bromo;

Cycloalkyl: monocyclic saturated hydrocarbon group having 3 to 7 ring atoms, such as cyclopropyl, cyclobutyl, cyclopentyl, cyclohexyl and cycloheptyl;

Alkoxycarbonyl: straight-chain or branched alkoxy groups having 1 to 6 carbon atoms (as mentioned above) which are attached to the skeleton via a carbonyl group (—CO—);

Aminothiocarbonyl: a —C(═S)NH₂ group;

Alkylsulfinyl: straight-chain or branched alkyl groups having 1 to 6 carbon atoms (as mentioned above) which are attached to the skeleton via a sulfinyl group (—SO—);

Alkylsulfonyl: straight-chain or branched alkyl groups having 1 to 6 carbon atoms (as mentioned above) which are attached to the skeleton via a sulfonyl group (—SO₂—);

With respect to the intended use of the fluoroalkene derivatives of formula I, particular preference is given to the following meanings of the substituents, in each case on their own or in combination:

Preference is given to compounds of formula I wherein R¹ is C₁-C₆-alkyl.

Particular preference is given to compounds of formula I wherein R¹ is methyl or ethyl.

Preference is furthermore given to compounds of formula I wherein R² is halogen, C₁-C₆-alkyl, or C₁-C₆-haloalkyl.

Particular preference is given to compounds of formula I wherein R² is halogen, preferably chloro or bromo.

Most preferred are compounds of formula I wherein R² is geminal chloro or bromo.

Moreover, preference is given to compounds of formula I wherein A is hydrogen, cyano, nitro or halogen.

Preference is further given to compounds of formula I wherein A is hydrogen, cyano, or halogen.

Particular preference is given to compounds of formula I wherein A is cyano.

Preference is given to compounds of formula I wherein B is hydrogen, halogen, Cₗ-C₆-alkoxy or C₁-C₆-alkylthio.

Particular preference is given to compounds of formula I wherein B is halogen.

Preference is given to compounds of formula I wherein Q is halogen.

Particular preference is given to compounds of formula I wherein Q is fluoro or chloro.

Preference is given to compounds of formula I wherein X is hydrogen or halogen.

Particular preference is given to compounds of formula I wherein X is hydrogen.

Preference is given to compounds of formula I wherein Y is halogen or C₁-C₆-haloalkyl.

Particular preference is given to compounds of formula I wherein Y is C₁-C₆-haloalkyl, especially trifluoromethyl.

Preference is given to compounds of formula I wherein Z is hydrogen or halogen.

Particular preference is given to compounds of formula I wherein Z is hydrogen.

Preference is given to compounds of formula I wherein M is nitrogen.

Likewise, preference is given to compounds of formula I wherein M is CR⁶.

Particular preference is given to compounds of formula I wherein M is CR⁶ and R⁶ is halogen, especially fluoro or chloro.

Preference is given to compounds of formula I wherein a) M is nitrogen and at least one of Q, X, Y, and Z is not hydrogen and b) M is CR⁶ and at least one of Q, X, Z and R⁶ is not hydrogen.

Preference is given to compounds of formula I wherein n is 1, 2, 3, or 4.

Particular preference is given to compounds of formula I wherein n is 1 or 2.

Particularly preferred compounds of the invention are those compounds of formula I wherein

Q is halogen,

Y is halogen or C₁-C₄-haloalkyl,

M is CR⁶ and

R⁶ is halogen.

Particular preference is also given to compounds of the invention are those compounds of formula I wherein

R¹ is C₁-C₄-alkyl,

R² is halogen,

Q is halogen,

Y is halogen or C₁-C₄-haloalkyl,

M is CR⁶ and

R⁶ is halogen.

Moreover, particular preference is given to compounds of the invention are those compounds of formula I wherein

R¹ is C₁-C₄-alkyl,

R² is halogen,

A is hydrogen, cyano, or halogen,

B is hydrogen, halogen, C₁-C₄-alkoxy, or C₁-C₄-alkylthio,

Q is halogen,

Y is halogen or C₁-C₄-haloalkyl,

M is CR⁶ and

R⁶ is halogen.

With respect to their use, particular preference is given to the compounds I.1 compiled in the Tables below. Moreover, the groups mentioned for a substituent in the Tables are on their own, independently of the combination in which they are mentioned, a particularly preferred embodiment of the substituent in question.

Depending on the substitution pattern of the cyclopropyl ring, the compounds of the Tables below can contain one or two chiral centers at the carbon atoms marked 2 or 3, in which case the respective enantiomers and diastereomers represent preferred compounds of the present invention.

Table 1

Compounds of formula I.1 wherein R¹ is methyl, R² is 2-chloro, n is 1, M is C—Cl and the combination of A, B, Q and Y for a compound corresponds in each case to a row of Table A.

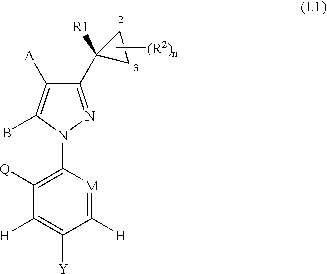

Table 2

Compounds of formula I.1 wherein R¹ is ethyl, R² is 2-chloro, n is 1, M is C—Cl and the combination of A, B, Q and Y for a compound corresponds in each case to a row of Table A.

Table 3

Compounds of formula I.1 wherein R¹ is hydrogen, R² is 2-chloro, n is 1, M is C—Cl and the combination of A, B, Q and Y for a compound corresponds in each case to a row of Table A.

Table 4

Compounds of formula I.1 wherein R¹ is methyl, R² is 3-chloro, n is 1, M is C—Cl and the combination of A, B, Q and Y for a compound corresponds in each case to a row of Table A.

Table 5

Compounds of formula I.1 wherein R¹ is ethyl, R² is 3-chloro, n is 1, M is C—Cl and the combination of A, B, Q and Y for a compound corresponds in each case to a row of Table A.

Table 6

Compounds of formula I.1 wherein R¹ is hydrogen, R² is 3-chloro, n is 1, M is C—Cl and the combination of A, B, Q and Y for a compound corresponds in each case to a row of Table A.

Table 7

Compounds of formula I.1 wherein R¹ is methyl, R² is 2-bromo, n is 1, M is C—Cl and the combination of A, B, Q and Y for a compound corresponds in each case to a row of Table A.

Table 8

Compounds of formula I.1 wherein R¹ is ethyl, R² is 2-bromo, n is 1, M is C—Cl and the combination of A, B, Q and Y for a compound corresponds in each case to a row of Table A.

Table 9

Compounds of formula I.1 wherein R¹ is hydrogen, R² is 2-bromo, n is 1, M is C—Cl and the combination of A, B, Q and Y for a compound corresponds in each case to a row of Table A.

Table 10

Compounds of formula I.1 wherein R¹ is methyl, R² is 3-bromo, n is 1, M is C—Cl and the combination of A, B, Q and Y for a compound corresponds in each case to a row of Table A.

Table 11

Compounds of formula I.1 wherein R¹ is ethyl, R² is 3-bromo, n is 1, M is C—Cl and the combination of A, B, Q and Y for a compound corresponds in each case to a row of Table A.

Table 12

Compounds of formula I.1 wherein R¹ is hydrogen, R² is 3-bromo, n is 1, M is C—Cl and the combination of A, B, Q and Y for a compound corresponds in each case to a row of Table A.

Table 13

Compounds of formula I.1 wherein R¹ is methyl, R² is 2,2-dichloro, n is 2, M is C—Cl and the combination of A, B, Q and Y for a compound corresponds in each case to a row of Table A.

Table 14

Compounds of formula I.1 wherein R¹ is ethyl, R² is 2,2-dichloro, n is 2, M is C—Cl and the combination of A, B, Q and Y for a compound corresponds in each case to a row of Table A.

Table 15

Compounds of formula I.1 wherein R¹ is hydrogen, R² is 2,2-dichloro, n is 1, M is C—Cl and the combination of A, B, Q and Y for a compound corresponds in each case to a row of Table A.

Table 16

Compounds of formula I.1 wherein R¹ is methyl, R² is 3,3-dichloro, n is 2, M is C—Cl and the combination of A, B, Q and Y for a compound corresponds in each case to a row of Table A.

Table 17

Compounds of formula I.1 wherein R¹ is ethyl, R² is 3,3-dichloro, n is 2, M is C—Cl and the combination of A, B, Q and Y for a compound corresponds in each case to a row of Table A.

Table 18

Compounds of formula I.1 wherein R¹ is hydrogen, R² is 3,3-dichloro, n is 1, M is C—Cl and the combination of A, B, Q and Y for a compound corresponds in each case to a row of Table A.

Table 19

Compounds of formula I.1 wherein R¹ is methyl, R² is 2,2-dibromo, n is 2, M is C—Cl and the combination of A, B, Q and Y for a compound corresponds in each case to a row of Table A.

Table 20

Compounds of formula I.1 wherein R¹ is ethyl, R² is 2,2-dibromo, n is 2, M is C—Cl and the combination of A, B, Q and Y for a compound corresponds in each case to a row of Table A.

Table 21

Compounds of formula I.1 wherein R¹ is hydrogen, R² is 2,2-dibromo, n is 2, M is C—Cl and the combination of A, B, Q and Y for a compound corresponds in each case to a row of Table A.

Table 22

Compounds of formula I.1 wherein R¹ is methyl, R² is 3,3-dibromo, n is 2, M is C—Cl and the combination of A, B, Q and Y for a compound corresponds in each case to a row of Table A.

Table 23

Compounds of formula I.1 wherein R¹ is ethyl, R² is 3,3-dibromo, n is 2, M is C—Cl and the combination of A, B, Q and Y for a compound corresponds in each case to a row of Table A.

Table 24

Compounds of formula I.1 wherein R¹ is hydrogen, R² is 3,3-dibromo, n is 2, M is C—Cl and the combination of A, B, Q and Y for a compound corresponds in each case to a row of Table A.

Table 25

Compounds of formula I.1 wherein R¹ is methyl, R² is 2-chloro, 3-methyl, n is 2, M is C—Cl and the combination of A, B, Q and Y for a compound corresponds in each case to a row of Table A.

Table 26

Compounds of formula I.1 wherein R¹ is ethyl, R² is 2-chloro, 3-methyl, n is 2, M is C—Cl and the combination of A, B, Q and Y for a compound corresponds in each case to a row of Table A.

Table 27

Compounds of formula I.1 wherein R¹ is hydrogen, R² is 2-chloro, 3-methyl, n is 2, M is C—Cl and the combination of A, B, Q and Y for a compound corresponds in each case to a row of Table A.

Table 28

Compounds of formula I.1 wherein R¹ is methyl, R² is 3-chloro, 2-methyl, n is 2, M is C—Cl and the combination of A, B, Q and Y for a compound corresponds in each case to a row of Table A.

Table 29

Compounds of formula I.1 wherein R¹ is ethyl, R² is 3-chloro, 2-methyl, n is 2, M is C—Cl and the combination of A, B, Q and Y for a compound corresponds in each case to a row of Table A.

Table 30

Compounds of formula I.1 wherein R¹ is hydrogen, R² is 3-chloro, 2-methyl, n is 2, M is C—Cl and the combination of A, B, Q and Y for a compound corresponds in each case to a row of Table A.

Table 31

Compounds of formula I.1 wherein R¹ is methyl, R² is 2-bromo, 3-methyl, n is 2, M is C—Cl and the combination of A, B, Q and Y for a compound corresponds in each case to a row of Table A.

Table 32

Compounds of formula I.1 wherein R¹ is ethyl, R² is 2-bromo, 3-methyl, n is 2, M is C—Cl and the combination of A, B, Q and Y for a compound corresponds in each case to a row of Table A.

Table 33

Compounds of formula I.1 wherein R¹ is hydrogen, R² is 2-bromo, 3-methyl, n is 2, M is C—Cl and the combination of A, B, Q and Y for a compound corresponds in each case to a row of Table A.

Table 34

Compounds of formula I.1 wherein R¹ is methyl, R² is 3-bromo, 2-methyl, n is 2, M is C—Cl and the combination of A, B, Q and Y for a compound corresponds in each case to a row of Table A.

Table 35

Compounds of formula I.1 wherein R¹ is ethyl, R² is 3-bromo, 2-methyl, n is 2, M is C—Cl and the combination of A, B, Q and Y for a compound corresponds in each case to a row of Table A.

Table 36

Compounds of formula I.1 wherein R¹ is hydrogen, R² is 3-bromo, 2-methyl, n is 2, M is C—Cl and the combination of A, B, Q and Y for a compound corresponds in each case to a row of Table A.

Table 37

Compounds of formula I.1 wherein R¹ is methyl, R² is 2,2-dichloro, 3-methyl, n is 3, M is C—Cl and the combination of A, B, Q and Y for a compound corresponds in each case to a row of Table A.

Table 38

Compounds of formula I.1 wherein R¹ is ethyl, R² is 2,2-dichloro, 3-methyl, n is 3, M is C—Cl and the combination of A, B, Q and Y for a compound corresponds in each case to a row of Table A.

Table 39

Compounds of formula I.1 wherein R¹ is hydrogen, R² is 2,2-dichloro, 3-methyl, n is 3, M is C—Cl and the combination of A, B, Q and Y for a compound corresponds in each case to a row of Table A.

Table 40

Compounds of formula I.1 wherein R¹ is methyl, R² is 3,3-dichloro, 2-methyl, n is 3, M is C—Cl and the combination of A, B, Q and Y for a compound corresponds in each case to a row of Table A.

Table 41

Compounds of formula I.1 wherein R¹ is ethyl, R² is 3,3-dichloro, 2-methyl, n is 3, M is C—Cl and the combination of A, B, Q and Y for a compound corresponds in each case to a row of Table A.

Table 42

Compounds of formula I.1 wherein R¹ is hydrogen, R² is 3,3-dichloro, 2-methyl, n is 3, M is C—Cl and the combination of A, B, Q and Y for a compound corresponds in each case to a row of Table A.

Table 43

Compounds of formula I.1 wherein R¹ is methyl, R² is 2,2-dibromo, 3-methyl, n is 3, M is C—Cl and the combination of A, B, Q and Y for a compound corresponds in each case to a row of Table A.

Table 44

Compounds of formula I.1 wherein R¹ is ethyl, R² is 2,2-dibromo, 3-methyl, n is 3, M is C—Cl and the combination of A, B, Q and Y for a compound corresponds in each case to a row of Table A.

Table 45

Compounds of formula I.1 wherein R¹ is hydrogen, R² is 2,2-dibromo, 3-methyl, n is 3, M is C—Cl and the combination of A, B, Q and Y for a compound corresponds in each case to a row of Table A.

Table 46

Compounds of formula I.1 wherein R¹ is methyl, R² is 3,3-dibromo, 2-methyl, n is 3, M is C—Cl and the combination of A, B, Q and Y for a compound corresponds in each case to a row of Table A.

Table 47

Compounds of formula I.1 wherein R¹ is ethyl, R² is 3,3-dibromo, 2-methyl, n is 3, M is C—Cl and the combination of A, B, Q and Y for a compound corresponds in each case to a row of Table A.

Table 48

Compounds of formula I.1 wherein R¹ is hydrogen, R² is 3,3-dibromo, 2-methyl, n is 3, M is C—Cl and the combination of A, B, Q and Y for a compound corresponds in each case to a row of Table A.

Table 49

Compounds of formula I.1 wherein R¹ is methyl, R² is 2-chloro, n is 1, M is C—F and the combination of A, B, Q and Y for a compound corresponds in each case to a row of Table A.

Table 50

Compounds of formula I.1 wherein R¹ is ethyl, R² is 2-chloro, n is 1, M is C—F and the combination of A, B, Q and Y for a compound corresponds in each case to a row of Table A.

Table 51

Compounds of formula I.1 wherein R¹ is hydrogen, R² is 2-chloro, n is 1, M is C—F and the combination of A, B, Q and Y for a compound corresponds in each case to a row of Table A.

Table 52

Compounds of formula I.1 wherein R¹ is methyl, R² is 3-chloro, n is 1, M is C—F and the combination of A, B, Q and Y for a compound corresponds in each case to a row of Table A.

Table 53

Compounds of formula I.1 wherein R¹ is ethyl, R² is 3-chloro, n is 1, M is C—F and the combination of A, B, Q and Y for a compound corresponds in each case to a row of Table A.

Table 54

Compounds of formula I.1 wherein R¹ is hydrogen, R² is 3-chloro, n is 1, M is C—F and the combination of A, B, Q and Y for a compound corresponds in each case to a row of Table A.

Table 55

Compounds of formula I.1 wherein R¹ is methyl, R² is 2-bromo, n is 1, M is C—F and the combination of A, B, Q and Y for a compound corresponds in each case to a row of Table A.

Table 56

Compounds of formula I.1 wherein R¹ is ethyl, R² is 2-bromo, n is 1, M is C—F and the combination of A, B, Q and Y for a compound corresponds in each case to a row of Table A.

Table 57

Compounds of formula I.1 wherein R¹ is hydrogen, R² is 2-bromo, n is 1, M is C—F and the combination of A, B, Q and Y for a compound corresponds in each case to a row of Table A.

Table 58

Compounds of formula I.1 wherein R¹ is methyl, R² is 3-bromo, n is 1, M is C—F and the combination of A, B, Q and Y for a compound corresponds in each case to a row of Table A.

Table 59

Compounds of formula I.1 wherein R¹ is ethyl, R² is 3-bromo, n is 1, M is C—F and the combination of A, B, Q and Y for a compound corresponds in each case to a row of Table A.

Table 60

Compounds of formula I.1 wherein R¹ is hydrogen, R² is 3-bromo, n is 1, M is C—F and the combination of A, B, Q and Y for a compound corresponds in each case to a row of Table A.

Table 61

Compounds of formula I.1 wherein R¹ is methyl, R² is 2,2-dichloro, n is 2, M is C—F and the combination of A, B, Q and Y for a compound corresponds in each case to a row of Table A.

Table 62

Compounds of formula I.1 wherein R¹ is ethyl, R² is 2,2-dichloro, n is 2, M is C—F and the combination of A, B, Q and Y for a compound corresponds in each case to a row of Table A.

Table 63

Compounds of formula I.1 wherein R¹ is hydrogen, R² is 2,2-dichloro, n is 2, M is C—F and the combination of A, B, Q and Y for a compound corresponds in each case to a row of Table A.

Table 64

Compounds of formula I.1 wherein R¹ is methyl, R² is 3,3-dichloro, n is 2, M is C—F and the combination of A, B, Q and Y for a compound corresponds in each case to a row of Table A.

Table 65

Compounds of formula I.1 wherein R¹ is ethyl, R² is 3,3-dichloro, n is 2, M is C—F and the combination of A, B, Q and Y for a compound corresponds in each case to a row of Table A.

Table 66

Compounds of formula I.1 wherein R¹ is hydrogen, R² is 3,3-dichloro, n is 2, M is C—F and the combination of A, B, Q and Y for a compound corresponds in each case to a row of Table A.

Table 67

Compounds of formula I.1 wherein R¹ is methyl, R² is 2,2-dibromo, n is 2, M is C—F and the combination of A, B, Q and Y for a compound corresponds in each case to a row of Table A.

Table 68

Compounds of formula I.1 wherein R¹ is ethyl, R² is 2,2-dibromo, n is 2, M is C—F and the combination of A, B, Q and Y for a compound corresponds in each case to a row of Table A.

Table 69

Compounds of formula I.1 wherein R¹ is hydrogen, R² is 2,2-dibromo, n is 2, M is C—F and the combination of A, B, Q and Y for a compound corresponds in each case to a row of Table A.

Table 70

Compounds of formula I.1 wherein R¹ is methyl, R² is 3,3-dibromo, n is 2, M is C—F and the combination of A, B, Q and Y for a compound corresponds in each case to a row of Table A.

Table 71

Compounds of formula I.1 wherein R¹ is ethyl, R² is 3,3-dibromo, n is 2, M is C—F and the combination of A, B, Q and Y for a compound corresponds in each case to a row of Table A.

Table 72

Compounds of formula I.1 wherein R¹ is hydrogen, R² is 3,3-dibromo, n is 2, M is C—F and the combination of A, B, Q and Y for a compound corresponds in each case to a row of Table A.

Table 73

Compounds of formula I.1 wherein R¹ is methyl, R² is 2-chloro, 3-methyl, n is 2, M is C—F and the combination of A, B, Q and Y for a compound corresponds in each case to a row of Table A.

Table 74

Compounds of formula I.1 wherein R¹ is ethyl, R² is 2-chloro, 3-methyl, n is 2, M is C—F and the combination of A, B, Q and Y for a compound corresponds in each case to a row of Table A.

Table 75

Compounds of formula I.1 wherein R¹ is hydrogen, R² is 2-chloro, 3-methyl, n is 2, M is C—F and the combination of A, B, Q and Y for a compound corresponds in each case to a row of Table A.

Table 76

Compounds of formula I.1 wherein R¹ is methyl, R² is 3-chloro, 2-methyl, n is 2, M is C—F and the combination of A, B, Q and Y for a compound corresponds in each case to a row of Table A.

Table 77

Compounds of formula I.1 wherein R¹ is ethyl, R² is 3-chloro, 2-methyl, n is 2, M is C—F and the combination of A, B, Q and Y for a compound corresponds in each case to a row of Table A.

Table 78

Compounds of formula I.1 wherein R¹ is hydrogen, R² is 3-chloro, 2-methyl, n is 2, M is C—F and the combination of A, B, Q and Y for a compound corresponds in each case to a row of Table A.

Table 79

Compounds of formula I.1 wherein R¹ is methyl, R² is 2-bromo, 3-methyl, n is 2, M is C—F and the combination of A, B, Q and Y for a compound corresponds in each case to a row of Table A.

Table 80

Compounds of formula I.1 wherein R¹ is ethyl, R² is 2-bromo, 3-methyl, n is 2, M is C—F and the combination of A, B, Q and Y for a compound corresponds in each case to a row of Table A.

Table 81

Compounds of formula I.1 wherein R¹ is hydrogen, R² is 2-bromo, 3-methyl, n is 2, M is C—F and the combination of A, B, Q and Y for a compound corresponds in each case to a row of Table A.

Table 82

Compounds of formula I.1 wherein R¹ is methyl, R² is 3-bromo, 2-methyl, n is 2, M is C—F and the combination of A, B, Q and Y for a compound corresponds in each case to a row of Table A.

Table 83

Compounds of formula I.1 wherein R¹ is ethyl, R² is 3-bromo, 2-methyl, n is 2, M is C—F and the combination of A, B, Q and Y for a compound corresponds in each case to a row of Table A.

Table 84

Compounds of formula I.1 wherein R¹ is hydrogen, R² is 3-bromo, 2-methyl, n is 2, M is C—F and the combination of A, B, Q and Y for a compound corresponds in each case to a row of Table A.

Table 85

Compounds of formula I.1 wherein R¹ is methyl, R² is 2,2-dichloro, 3-methyl, n is 3, M is C—F and the combination of A, B, Q and Y for a compound corresponds in each case to a row of Table A.

Table 86

Compounds of formula I.1 wherein R¹ is ethyl, R² is 2,2-dichloro, 3-methyl, n is 3, M is C—F and the combination of A, B, Q and Y for a compound corresponds in each case to a row of Table A.

Table 87

Compounds of formula I.1 wherein R¹ is hydrogen, R² is 2,2-dichloro, 3-methyl, n is 3, M is C—F and the combination of A, B, Q and Y for a compound corresponds in each case to a row of Table A.

Table 88

Compounds of formula I.1 wherein R¹ is methyl, R² is 3,3-dichloro, 2-methyl, n is 3, M is C—F and the combination of A, B, Q and Y for a compound corresponds in each case to a row of Table A.

Table 89

Compounds of formula I.1 wherein R¹ is ethyl, R² is 3,3-dichloro, 2-methyl, n is 3, M is C—F and the combination of A, B, Q and Y for a compound corresponds in each case to a row of Table A.

Table 90

Compounds of formula I.1 wherein R¹ is hydrogen, R² is 3,3-dichloro, 2-methyl, n is 3, M is C—F and the combination of A, B, Q and Y for a compound corresponds in each case to a row of Table A.

Table 91

Compounds of formula I.1 wherein R¹ is methyl, R² is 2,2-dibromo, 3-methyl, n is 3, M is C—F and the combination of A, B, Q and Y for a compound corresponds in each case to a row of Table A.

Table 92

Compounds of formula I.1 wherein R¹ is ethyl, R² is 2,2-dibromo, 3-methyl, n is 3, M is C—F and the combination of A, B, Q and Y for a compound corresponds in each case to a row of Table A.

Table 93

Compounds of formula I.1 wherein R¹ is hydrogen, R² is 2,2-dibromo, 3-methyl, n is 3, M is C—F and the combination of A, B, Q and Y for a compound corresponds in each case to a row of Table A.

Table 94

Compounds of formula I.1 wherein R¹ is methyl, R² is 3,3-dibromo, 2-methyl, n is 3, M is C—F and the combination of A, B, Q and Y for a compound corresponds in each case to a row of Table A.

Table 95

Compounds of formula I.1 wherein R¹ is ethyl, R² is 3,3-dibromo, 2-methyl, n is 3, M is C—F and the combination of A, B, Q and Y for a compound corresponds in each case to a row of Table A.

Table 96

Compounds of formula I.1 wherein R¹ is hydrogen, R² is 3,3-dibromo, 2-methyl, n is 3, M is C—F and the combination of A, B, Q and Y for a compound corresponds in each case to a row of Table A.

Table 97

Compounds of formula I.1 wherein R¹ is methyl, R² is 2-chloro, n is 1, M is N and the combination of A, B, Q and Y for a compound corresponds in each case to a row of Table A.

Table 98

Compounds of formula I.1 wherein R¹ is ethyl, R² is 2-chloro, n is 1, M is N and the combination of A, B, Q and Y for a compound corresponds in each case to a row of Table A.

Table 99

Compounds of formula I.1 wherein R¹ is hydrogen, R² is 2-chloro, n is 1, M is N and the combination of A, B, Q and Y for a compound corresponds in each case to a row of Table A.

Table 100

Compounds of formula I.1 wherein R¹ is methyl, R² is 3-chloro, n is 1, M is N and the combination of A, B, Q and Y for a compound corresponds in each case to a row of Table A.

Table 101

Compounds of formula I.1 wherein R¹ is ethyl, R² is 3-chloro, n is 1, M is N and the combination of A, B, Q and Y for a compound corresponds in each case to a row of Table A.

Table 102

Compounds of formula I.1 wherein R¹ is hydrogen, R² is 3-chloro, n is 1, M is N and the combination of A, B, Q and Y for a compound corresponds in each case to a row of Table A.

Table 103

Compounds of formula I.1 wherein R¹ is methyl, R² is 2-bromo, n is 1, M is N and the combination of A, B, Q and Y for a compound corresponds in each case to a row of Table A.

Table 104

Compounds of formula I.1 wherein R¹ is ethyl, R² is 2-bromo, n is 1, M is N and the combination of A, B, Q and Y for a compound corresponds in each case to a row of Table A.

Table 105

Compounds of formula I.1 wherein R¹ is hydrogen, R² is 2-bromo, n is 1, M is N and the combination of A, B, Q and Y for a compound corresponds in each case to a row of Table A.

Table 106

Compounds of formula I.1 wherein R¹ is methyl, R² is 3-bromo, n is 1, M is N and the combination of A, B, Q and Y for a compound corresponds in each case to a row of Table A.

Table 107

Compounds of formula I.1 wherein R¹ is ethyl, R² is 3-bromo, n is 1, M is N and the combination of A, B, Q and Y for a compound corresponds in each case to a row of Table A.

Table 108

Compounds of formula I.1 wherein R¹ is hydrogen, R² is 3-bromo, n is 1, M is N and the combination of A, B, Q and Y for a compound corresponds in each case to a row of Table A.

Table 109

Compounds of formula I.1 wherein R¹ is methyl, R² is 2,2-dichloro, n is 2, M is N and the combination of A, B, Q and Y for a compound corresponds in each case to a row of Table A.

Table 110

Compounds of formula I.1 wherein R¹ is ethyl, R² is 2,2-dichloro, n is 2, M is N and the combination of A, B, Q and Y for a compound corresponds in each case to a row of Table A.

Table 111

Compounds of formula I.1 wherein R¹ is hydrogen, R² is 2,2-dichloro, n is 2, M is N and the combination of A, B, Q and Y for a compound corresponds in each case to a row of Table A.

Table 112

Compounds of formula I.1 wherein R¹ is methyl, R² is 3,3-dichloro, n is 2, M is N and the combination of A, B, Q and Y for a compound corresponds in each case to a row of Table A.

Table 113

Compounds of formula I.1 wherein R¹ is ethyl, R² is 3,3-dichloro, n is 2, M is N and the combination of A, B, Q and Y for a compound corresponds in each case to a row of Table A.

Table 114

Compounds of formula I.1 wherein R¹ is hydrogen, R² is 3,3-dichloro, n is 2, M is N and the combination of A, B, Q and Y for a compound corresponds in each case to a row of Table A.

Table 115

Compounds of formula I.1 wherein R¹ is methyl, R² is 2,2-dibromo, n is 2, M is N and the combination of A, B, Q and Y for a compound corresponds in each case to a row of Table A.

Table 116

Compounds of formula I.1 wherein R¹ is ethyl, R² is 2,2-dibromo, n is 2, M is N and the combination of A, B, Q and Y for a compound corresponds in each case to a row of Table A.

Table 117

Compounds of formula I.1 wherein R¹ is hydrogen, R² is 2,2-dibromo, n is 2, M is N and the combination of A, B, Q and Y for a compound corresponds in each case to a row of Table A.

Table 118

Compounds of formula I.1 wherein R¹ is methyl, R² is 3,3-dibromo, n is 2, M is N and the combination of A, B, Q and Y for a compound corresponds in each case to a row of Table A.

Table 119

Compounds of formula I.1 wherein R¹ is ethyl, R² is 3,3-dibromo, n is 2, M is N and the combination of A, B, Q and Y for a compound corresponds in each case to a row of Table A.

Table 120

Compounds of formula I.1 wherein R¹ is hydrogen, R² is 3,3-dibromo, n is 2, M is N and the combination of A, B, Q and Y for a compound corresponds in each case to a row of Table A.

Table 121

Compounds of formula I.1 wherein R¹ is methyl, R² is 2-chloro, 3-methyl, n is 2, M is N and the combination of A, B, Q and Y for a compound corresponds in each case to a row of Table A.

Table 122

Compounds of formula I.1 wherein R¹ is ethyl, R² is 2-chloro, 3-methyl, n is 2, M is N and the combination of A, B, Q and Y for a compound corresponds in each case to a row of Table A.

Table 123

Compounds of formula I.1 wherein R¹ is hydrogen, R² is 2-chloro, 3-methyl, n is 2, M is N and the combination of A, B, Q and Y for a compound corresponds in each case to a row of Table A.

Table 124

Compounds of formula I.1 wherein R¹ is methyl, R² is 3-chloro, 2-methyl, n is 2, M is N and the combination of A, B, Q and Y for a compound corresponds in each case to a row of Table A.

Table 125

Compounds of formula I.1 wherein R¹ is ethyl, R² is 3-chloro, 2-methyl, n is 2, M is N and the combination of A, B, Q and Y for a compound corresponds in each case to a row of Table A.

Table 126

Compounds of formula I.1 wherein R¹ is hydrogen, R² is 3-chloro, 2-methyl, n is 2, M is N and the combination of A, B, Q and Y for a compound corresponds in each case to a row of Table A.

Table 127

Compounds of formula I.1 wherein R¹ is methyl, R² is 2-bromo, 3-methyl, n is 2, M is N and the combination of A, B, Q and Y for a compound corresponds in each case to a row of Table A.

Table 128

Compounds of formula I.1 wherein R¹ is ethyl, R² is 2-bromo, 3-methyl, n is 2, M is N and the combination of A, B, Q and Y for a compound corresponds in each case to a row of Table A.

Table 129

Compounds of formula I.1 wherein R¹ is hydrogen, R² is 2-bromo, 3-methyl, n is 2, M is N and the combination of A, B, Q and Y for a compound corresponds in each case to a row of Table A.

Table 130

Compounds of formula I.1 wherein R¹ is methyl, R² is 3-bromo, 2-methyl, n is 2, M is N and the combination of A, B, Q and Y for a compound corresponds in each case to a row of Table A.

Table 131

Compounds of formula I.1 wherein R¹ is ethyl, R² is 3-bromo, 2-methyl, n is 2, M is N and the combination of A, B, Q and Y for a compound corresponds in each case to a row of Table A.

Table 132

Compounds of formula I.1 wherein R¹ is hydrogen, R² is 3-bromo, 2-methyl, n is 2, M is N and the combination of A, B, Q and Y for a compound corresponds in each case to a row of Table A.

Table 133

Compounds of formula I.1 wherein R¹ is methyl, R² is 2,2-dichloro, 3-methyl, n is 3, M is N and the combination of A, B, Q and Y for a compound corresponds in each case to a row of Table A.

Table 134

Compounds of formula I.1 wherein R¹ is ethyl, R² is 2,2-dichloro, 3-methyl, n is 3, M is N and the combination of A, B, Q and Y for a compound corresponds in each case to a row of Table A.

Table 135

Compounds of formula I.1 wherein R¹ is hydrogen, R² is 2,2-dichloro, 3-methyl, n is 3, M is N and the combination of A, B, Q and Y for a compound corresponds in each case to a row of Table A.

Table 136

Compounds of formula I.1 wherein R¹ is methyl, R² is 3,3-dichloro, 2-methyl, n is 3, M is N and the combination of A, B, Q and Y for a compound corresponds in each case to a row of Table A.

Table 137

Compounds of formula I.1 wherein R¹ is ethyl, R² is 3,3-dichloro, 2-methyl, n is 3, M is N and the combination of A, B, Q and Y for a compound corresponds in each case to a row of Table A.

Table 138

Compounds of formula I.1 wherein R¹ is hydrogen, R² is 3,3-dichloro, 2-methyl, n is 3, M is N and the combination of A, B, Q and Y for a compound corresponds in each case to a row of Table A.

Table 139

Compounds of formula I.1 wherein R¹ is methyl, R² is 2,2-dibromo, 3-methyl, n is 3, M is N and the combination of A, B, Q and Y for a compound corresponds in each case to a row of Table A.

Table 140

Compounds of formula I.1 wherein R¹ is ethyl, R² is 2,2-dibromo, 3-methyl, n is 3, M is N and the combination of A, B, Q and Y for a compound corresponds in each case to a row of Table A.

Table 141

Compounds of formula I.1 wherein R¹ is hydrogen, R² is 2,2-dibromo, 3-methyl, n is 3, M is N and the combination of A, B, Q and Y for a compound corresponds in each case to a row of Table A.

Table 142

Compounds of formula I.1 wherein R¹ is methyl, R² is 3,3-dibromo, 2-methyl, n is 3, M is N and the combination of A, B, Q and Y for a compound corresponds in each case to a row of Table A.

Table 143

Compounds of formula I.1 wherein R¹ is ethyl, R² is 3,3-dibromo, 2-methyl, n is 3, M is N and the combination of A, B, Q and Y for a compound corresponds in each case to a row of Table A.

Table 144

Compounds of formula I.1 wherein R¹ is hydrogen, R² is 3,3-dibromo, 2-methyl, n is 3, M is N and the combination of A, B, Q and Y for a compound corresponds in each case to a row of Table A.

TABLE A

(I.1)

| Nr. | A | B | Q | Y |
|---|---|---|---|---|
| A-1 | H | H | Cl | Cl |
| A-2 | CN | H | Cl | Cl |
| A-3 | Cl | H | Cl | Cl |
| A-4 | Br | H | Cl | Cl |
| A-5 | NO₂ | H | Cl | Cl |
| A-6 | H | Cl | Cl | Cl |
| A-7 | CN | Cl | Cl | Cl |
| A-8 | Cl | Cl | Cl | Cl |
| A-9 | Br | Cl | Cl | Cl |
| A-10 | NO₂ | Cl | Cl | Cl |
| A-11 | H | Br | Cl | Cl |
| A-12 | CN | Br | Cl | Cl |
| A-13 | Cl | Br | Cl | Cl |
| A-14 | Br | Br | Cl | Cl |
| A-15 | NO₂ | Br | Cl | Cl |
| A-16 | H | I | Cl | Cl |
| A-17 | CN | I | Cl | Cl |
| A-18 | Cl | I | Cl | Cl |
| A-19 | Br | I | Cl | Cl |
| A-20 | NO₂ | I | Cl | Cl |
| A-21 | H | OCHF₂ | Cl | Cl |
| A-22 | CN | OCHF₂ | Cl | Cl |
| A-23 | Cl | OCHF₂ | Cl | Cl |
| A-24 | Br | OCHF₂ | Cl | Cl |
| A-25 | NO₂ | OCHF₂ | Cl | Cl |
| A-26 | H | OCH₃ | Cl | Cl |
| A-27 | CN | OCH₃ | Cl | Cl |
| A-28 | Cl | OCH₃ | Cl | Cl |
| A-29 | Br | OCH₃ | Cl | Cl |
| A-30 | NO₂ | OCH₃ | Cl | Cl |
| A-31 | H | H | F | Cl |
| A-32 | CN | H | F | Cl |
| A-33 | Cl | H | F | Cl |
| A-34 | Br | H | F | Cl |
| A-35 | NO₂ | H | F | Cl |
| A-36 | H | Cl | F | Cl |
| A-37 | CN | Cl | F | Cl |
| A-38 | Cl | Cl | F | Cl |
| A-39 | Br | Cl | F | Cl |
| A-40 | NO₂ | Cl | F | Cl |
| A-41 | H | Br | F | Cl |
| A-42 | CN | Br | F | Cl |
| A-43 | Cl | Br | F | Cl |
| A-44 | Br | Br | F | Cl |
| A-45 | NO₂ | Br | F | Cl |
| A-46 | H | I | F | Cl |
| A-47 | CN | I | F | Cl |
| A-48 | Cl | I | F | Cl |
| A-49 | Br | I | F | Cl |
| A-50 | NO₂ | I | F | Cl |
| A-51 | H | OCHF₂ | F | Cl |
| A-52 | CN | OCHF₂ | F | Cl |
| A-53 | Cl | OCHF₂ | F | Cl |
| A-54 | Br | OCHF₂ | F | Cl |
| A-55 | NO₂ | OCHF₂ | F | Cl |
| A-56 | H | OCH₃ | F | Cl |
| A-57 | CN | OCH₃ | F | Cl |
| A-58 | Cl | OCH₃ | F | Cl |
| A-59 | Br | OCH₃ | F | Cl |
| A-60 | NO₂ | OCH₃ | F | Cl |
| A-61 | H | H | Cl | F |
| A-62 | CN | H | Cl | F |
| A-63 | Cl | H | Cl | F |
| A-64 | Br | H | Cl | F |
| A-65 | NO₂ | H | Cl | F |
| A-66 | H | Cl | Cl | F |
| A-67 | CN | Cl | Cl | F |
| A-68 | Cl | Cl | Cl | F |
| A-69 | Br | Cl | Cl | F |
| A-70 | NO₂ | Cl | Cl | F |
| A-71 | H | Br | Cl | F |
| A-72 | CN | Br | Cl | F |
| A-73 | Cl | Br | Cl | F |
| A-74 | Br | Br | Cl | F |
| A-75 | NO₂ | Br | Cl | F |
| A-76 | H | I | Cl | F |
| A-77 | CN | I | Cl | F |
| A-78 | Cl | I | Cl | F |
| A-79 | Br | I | Cl | F |
| A-80 | NO₂ | I | Cl | F |
| A-81 | H | OCHF₂ | Cl | F |
| A-82 | CN | OCHF₂ | Cl | F |
| A-83 | Cl | OCHF₂ | Cl | F |
| A-84 | Br | OCHF₂ | Cl | F |
| A-85 | NO₂ | OCHF₂ | Cl | F |
| A-86 | H | OCH₃ | Cl | F |
| A-87 | CN | OCH₃ | Cl | F |
| A-88 | Cl | OCH₃ | Cl | F |
| A-89 | Br | OCH₃ | Cl | F |
| A-90 | NO₂ | OCH₃ | Cl | F |
| A-91 | H | H | F | F |
| A-92 | CN | H | F | F |
| A-93 | Cl | H | F | F |
| A-94 | Br | H | F | F |
| A-95 | NO₂ | H | F | F |
| A-96 | H | Cl | F | F |
| A-97 | CN | Cl | F | F |
| A-98 | Cl | Cl | F | F |
| A-99 | Br | Cl | F | F |
| A-100 | NO₂ | Cl | F | F |
| A-101 | H | Br | F | F |
| A-102 | CN | Br | F | F |
| A-103 | Cl | Br | F | F |
| A-104 | Br | Br | F | F |
| A-105 | NO₂ | Br | F | F |
| A-106 | H | I | F | F |
| A-107 | CN | I | F | F |
| A-108 | Cl | I | F | F |
| A-109 | Br | I | F | F |
| A-110 | NO₂ | I | F | F |
| A-111 | H | OCHF₂ | F | F |
| A-112 | CN | OCHF₂ | F | F |
| A-113 | Cl | OCHF₂ | F | F |
| A-114 | Br | OCHF₂ | F | F |
| A-115 | NO₂ | OCHF₂ | F | F |
| A-116 | H | OCH₃ | F | F |
| A-117 | CN | OCH₃ | F | F |
| A-118 | Cl | OCH₃ | F | F |
| A-119 | Br | OCH₃ | F | F |
| A-120 | NO₂ | OCH₃ | F | F |
| A-121 | H | H | Cl | CF₃ |
| A-122 | CN | H | Cl | CF₃ |
| A-123 | Cl | H | Cl | CF₃ |
| A-124 | Br | H | Cl | CF₃ |
| A-125 | NO₂ | H | Cl | CF₃ |
| A-126 | H | Cl | Cl | CF₃ |
| A-127 | CN | Cl | Cl | CF₃ |
| A-128 | Cl | Cl | Cl | CF₃ |
| A-129 | Br | Cl | Cl | CF₃ |
| A-130 | NO₂ | Cl | Cl | CF₃ |
| A-131 | H | Br | Cl | CF₃ |
| A-132 | CN | Br | Cl | CF₃ |
| A-133 | Cl | Br | Cl | CF₃ |
| A-134 | Br | Br | Cl | CF₃ |
| A-135 | NO₂ | Br | Cl | CF₃ |
| A-136 | H | I | Cl | CF₃ |
| A-137 | CN | I | Cl | CF₃ |
| A-138 | Cl | I | Cl | CF₃ |
| A-139 | Br | I | Cl | CF₃ |
| A-140 | NO₂ | I | Cl | CF₃ |
| A-141 | H | OCHF₂ | Cl | CF₃ |
| A-142 | CN | OCHF₂ | Cl | CF₃ |
| A-143 | Cl | OCHF₂ | Cl | CF₃ |
| A-144 | Br | OCHF₂ | Cl | CF₃ |
| A-145 | NO₂ | OCHF₂ | Cl | CF₃ |
| A-146 | H | OCH₃ | Cl | CF₃ |
| A-147 | CN | OCH₃ | Cl | CF₃ |
| A-148 | Cl | OCH₃ | Cl | CF₃ |
| A-149 | Br | OCH₃ | Cl | CF₃ |
| A-150 | NO₂ | OCH₃ | Cl | CF₃ |
| A-151 | H | H | F | CF₃ |
| A-152 | CN | H | F | CF₃ |
| A-153 | Cl | H | F | CF₃ |
| A-154 | Br | H | F | CF₃ |
| A-155 | NO₂ | H | F | CF₃ |
| A-156 | H | Cl | F | CF₃ |
| A-157 | CN | Cl | F | CF₃ |
| A-158 | Cl | Cl | F | CF₃ |
| A-159 | Br | Cl | F | CF₃ |
| A-160 | NO₂ | Cl | F | CF₃ |
| A-161 | H | Br | F | CF₃ |
| A-162 | CN | Br | F | CF₃ |
| A-163 | Cl | Br | F | CF₃ |
| A-164 | Br | Br | F | CF₃ |
| A-165 | NO₂ | Br | F | CF₃ |
| A-166 | H | I | F | CF₃ |
| A-167 | CN | I | F | CF₃ |
| A-168 | Cl | I | F | CF₃ |
| A-169 | Br | I | F | CF₃ |
| A-170 | NO₂ | I | F | CF₃ |
| A-171 | H | OCHF₂ | F | CF₃ |
| A-172 | CN | OCHF₂ | F | CF₃ |
| A-173 | Cl | OCHF₂ | F | CF₃ |
| A-174 | Br | OCHF₂ | F | CF₃ |
| A-175 | NO₂ | OCHF₂ | F | CF₃ |
| A-176 | H | OCH₃ | F | CF₃ |
| A-177 | CN | OCH₃ | F | CF₃ |
| A-178 | Cl | OCH₃ | F | CF₃ |
| A-179 | Br | OCH₃ | F | CF₃ |
| A-180 | NO₂ | OCH₃ | F | CF₃ |

Preferably, compounds of formula Ia wherein B is hydrogen, A is cyano and the other variables and the index are as defined for formula I are obtainable by reacting a hydrazonyl chloride of formula II wherein the variables and the index are as defined for formula I, with fumaronitrile in the presence of a base.

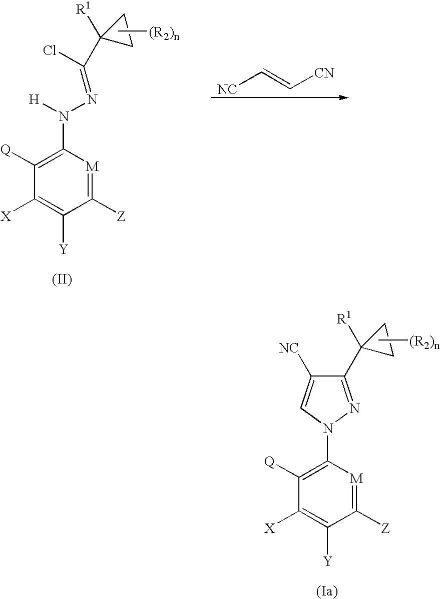

The reaction is usually carried out at temperatures of from 0° C. to 100° C., preferably from 10° C. to 30° C., in an inert organic solvent in the presence of a base.

Suitable solvents are aliphatic hydrocarbons, aromatic hydrocarbons, halogenated hydrocarbons, ethers, such as diethylether, diisopropylether, tert.-butylmethylether, digylme, dioxane, anisol and tetrahydrofuran, nitriles, ketones, alcoholes and also dimethyl sulfoxide, dimethyl formamide and dimethyl acetamide. Preferred solvents are tetrahydrofuran and dimethyl formamide. It is also possible to use mixtures of the solvents mentioned.

Suitable bases are inorganic compounds, such as alkali metal and alkaline earth metal hydroxides, alkali metal and alkaline earth metal carbonates, alkali metal bicarbonates, alkali metal and earth alkali metal alcoholates, and also organic bases, such as tertiary amines, such as trimethyl amine, triethyl amine, tri-isopropyl ethyl amine, N-methyl-piperidine, and pyridine. Substituted pyridine are for example collidine, lutidine and 4-dimethyl amino pyridine as well as bicyclic amines. Particular preference is given to tertiary amines, especially triethyl amine.

Fumaronitrile is commercially available.

Hydrazonyl chlorides of formula II may be prepared by conventional methods such as reacting in a first step a carboxyl derivative of formula III wherein the variables and the index are as defined for formula I and L is a nucleophilically exchangeable leaving group such as halogen, e.g. chloro or bromo, hetaryl, e.g. imidazolyl or pyridyl, carboxylate, e.g. acetat or trifluoroacetat, or sulfonate, e.g. mesylate or triflate, with a hydrazine of formula IV wherein the variables are as defined for formula I, and treating the resulting formula V hydrazide with a chlorinating agent, such as thionyl chloride.

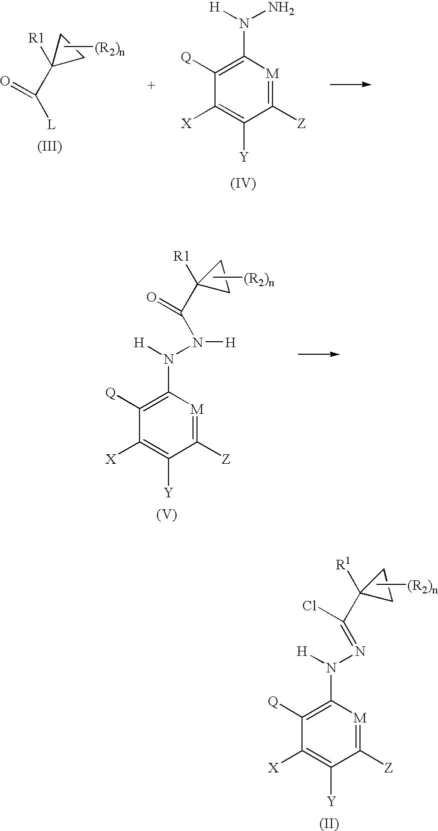

The first reaction step, the reaction of compounds III with compounds IV, is usually carried out at temperatures of from 0° C. to the boiling point of the reaction mixture in an inert organic solvent, optionally in the presence of a base [lit.: Houben-Weyl, “Methoden der Organischen Chemie”, 4. Auflage, Band X/2, Georg Thieme Verlag Stuttgart 1989, pp 349].

Compounds III can be used directly, as in the case of the alkyl-halogenides and carboxylic acid halogenides, sulfonic acid halogenides, carboxylic acid anhydrides, or they can be prepared in situ, e.g. in form of the activated carboxylic acids, prepared from the carboxylic acid and dicyclohexylcarbodiimide, carbonyldiimidazole, or 1-(3-dimethylaminopropyl)-3-ethyl-carbodiimide.

Suitable solvents are halogenated hydrocarbons, such as methylene chloride, chloroform and chlorobenzene, aromatic hydrocarbons such as toluolene, o-, m- and p-xylene, or chlorobenzene, ethers, such as diethylether, diisopropylether, tert.-butylmethylether, digylme, dioxane, anisol and tetrahydrofuran, polar aprotic solvents such as acetonitrile, proprionitrile, dimethyl sulfoxide, dimethyl formamide and dimethyl acetamide, or ester, such as acetic acid ethylester. It is also possible to use mixtures of the solvents mentioned.

Suitable bases are inorganic compounds, such as alkali metal and alkaline earth metal hydrides, e.g. sodium hydride, or alkali metal and alkaline earth metal carbonates, such as lithium carbonate or sodium carbonate, or organic bases, such as tertiary amines, such as trimethyl amine, triethyl amine, tri-isopropyl ethyl amine, N-methyl-piperidine, and pyridine. Substituted pyridine are for example collidine, lutidine and 4-dimethyl amino pyridine as well as bicyclic amines. Particular preference is given to triethyl amine and pyridine.

In general, the base is employed in equimolar amounts or in excess.

The starting materials are generally reacted with one another in equimolar amounts. In terms of yield, it may be advantageous to use an excess of one of the starting compounds.

Carboxyl derivatives of formula III are known, or they can be prepared by known methods [lit.: Aust. J. Chem. 1981, 34, 2461].

Hydrazines of formula IV are known from the literature or are commercially available, or they can be prepared by known methods [lit.: Houben-Weyl, “Methoden der organischen Chemie”, 4. Auflage, Band X/2, p 203].

The second reaction step, the chlorination of compounds V to compounds II, is usually carried out at temperatures of from 0° C. to 150° C., preferably from 80° C. to 120° C., in an inert organic solvent or in a chlorinating agent, preferably thionyl chloride [lit.: Houben-Weyl, “Methoden der Organischen Chemie”, 4. Auflage, Band X/2, p 378].

Suitable solvents are aliphatic hydrocarbons, aromatic hydrocarbons, or halogenated hydrocarbons.

The starting materials are generally reacted with one another in equimolar amounts. In terms of yield, it may be advantageous to use an excess of the chlorinating agent on compounds V.

Compounds of formula Ib wherein A is cyano, B is amino, and the further variables and the index are as defined for formula I, may be prepared by reacting a compound of formula II with malononitrile.

Diazotization of the formula Ib 5-aminopyrazoles with sodium nitrite in hydrochloric acid followed by halogenation with a halogenating agent, such as Cu halogenid of the chemical formula Cu-Hal gives the 5-halopyrazole of formula Ic wherein A is cyano, Hal is halogen, and the further variables and the index are as defined for formula I.

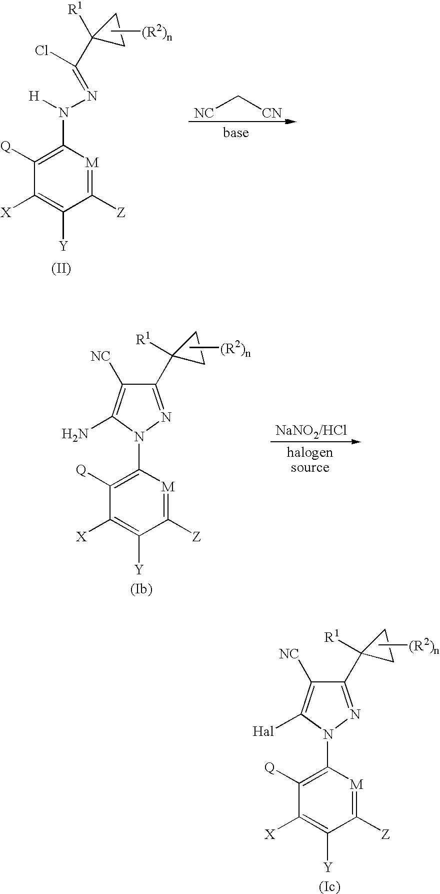

The reaction of compounds II with malononitrile is usually carried out at temperatures of from −10° C. to 100° C., preferably from 0° C. to 20° C., in an inert organic solvent in the presence of a base [lit.: J. Chem. Res., Synop. 1994, 6-7].

Suitable solvents are aliphatic hydrocarbons, aromatic hydrocarbons, halogenated hydrocarbons, ethers, such as diethylether, diisbpropylether, tert.-butylmethylether, digylme, dioxane, anisol and tetrahydrofuran, nitriles, and also dimethyl sulfoxide, dimethyl formamide and dimethyl acetamide. Preferred solvents are ethers, especially tetrahydrofuran. It is also possible to use mixtures of the solvents mentioned.

Suitable bases are inorganic compounds, such as alkali metal and alkaline earth metal hydroxides, alkali metal and alkaline earth metal oxides, alkali metal and alkaline earth metal hydrides, such as lithium hydride, sodium hydride, potassium hydride, and calcium hydride, alkali metal and alkaline earth metal amides, alkali metal and alkaline earth metal carbonates, alkali metal bicarbonates, organometallic compounds such as alkali metal alkyles, alkyl magnesium halogenides, alkali metal and earth alkali metal alcoholates, and also organic bases, such as tertiary amines. Particular preference is given to alkali metal hydrides, especially sodium hydride.

In general, the base is employed in catalytic amounts. It may however also be employed in equimolar amounts, in excess, or as a solvent.

The starting materials are generally reacted with one another in equimolar amounts. In terms of yield, it may be advantageous to use an excess of malononitrile based on compounds II.

Compounds of formula II are obtainable by the reaction described above. Malononitrile is commercially available.

The diazotation of compounds Ib followed by halogenation to yield compounds Ic is usually carried out without isolation of the intermediates.

The diazotation is usually carried out at temperatures of from −10° C. to 50° C., preferably from −5° C. to 5° C. The halogenation following the diazotation of compounds Ib to yield compounds Ic is carried out at temperatures of from 0° C. to 100° C., preferably from 20° C. to 80° C., in the presence of a halogen source [Lit: WO 97/07114 and literature cited therein].

The diazotation may be carried out in water or concentrated acids such as hydrochloric acid, hydrobromic acid, sulfuric acid, or perchloric acid, and also organic acids such as formic acid, acetic acid, and propionic acid. As halogen source, a transition metal halogenide such as a copper halogenide is added in aqueous solution.

The diazotation may also be carried out by reaction of compounds Ib with alkyl nitrites (alkyl-ONO) in an inert organic solvent. Suitable solvents are aromatic hydrocarbons, halogenated hydrocarbons, ethers and nitriles. In this case, bromine in chloroform or bromoform is used as halogen source.

The starting materials are generally reacted with one another in equimolar amounts. In terms of yield, it may be advantageous to use an excess of the halogen source on the diazotation product.

Compounds Ib can preferably be prepared by reaction of dicyanoalkene compounds of formula VI wherein the variables and the index are as defined for formula I and G is halogen, hydroxy, or alkoxy with hydrazines of formula V.

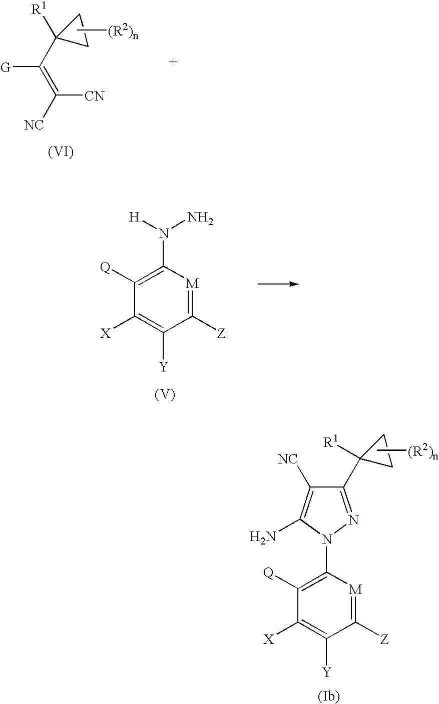

The reaction is usually carried out at temperatures of from 20° C. to 150° C., preferably from 50° C. to 100° C., in an inert organic solvent [Lit. e.g. WO 97/07114].

Suitable solvents are aliphatic hydrocarbons, aromatic hydrocarbons, halogenated hydrocarbons, ethers, nitriles, ketones, alcoholes such as methanol, ethanol, n-propanol, isopropanol, n-Butanol and tert.-butanol, and also dimethyl sulfoxide, dimethyl formamide and dimethyl acetamide. Preferred solvents are alcoholes such as ethanol. It is also possible to use mixtures of the solvents mentioned.

Dicyanoalkenes VI can be prepared under conditions known from WO 97/07114 and the literature cited therein. In a first step, reaction of carboxylic acid derivatives III′ wherein the variables and the index are as defined for formula I and L′ is carboxylate or halogen, such as chloro or bromo, with malononitrile to give compounds VI wherein G is hydroxy. Alkylation or halogenation, respectively, of enols VI′ gives compounds VI wherein G is alkoxy or halogen.

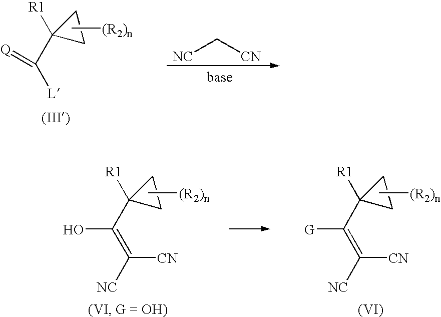

Carboxylic acid derivatives of formula III′ are known from the literature, or they can be prepared by known methods (compare above for formula III).

Compounds of formula Id wherein A is hydrogen, B is amino and any further variables and the index are as defined for formula I can be prepared by reacting a compound of formula VII wherein the variables and the index are as defined for formula I and L″ is alkoxy, amino, or dialkylamino, with a hydrazine of formula V.

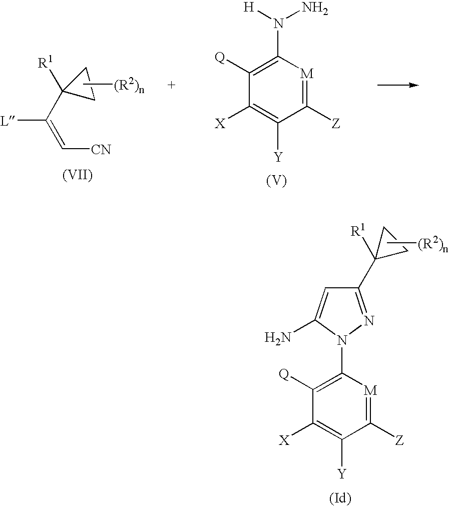

The reaction is usually carried out at temperatures of from 0° C. to 100° C., preferably from 20° C. to 80° C., in an inert organic solvent in the presence of an acid [lit. EP-A 679 650].

Suitable solvents are aliphatic hydrocarbons, aromatic hydrocarbons, halogenated hydrocarbons, alcoholes such as methanol, ethanol, n-propanol, isopropanol, n-butanol and tert.-butanol, and also dimethyl sulfoxide, dimethyl formamide and dimethyl acetamide. Preferred solvents alcoholes such as ethanol. It is also possible to use mixtures of the solvents mentioned.

Suitable acids or acid catalysts are inorganic acids such as hydrofluoric acid, hydrochloric acid, hydrobromic acid, sulfuric acid and perchloric acid, Lewis-acids, such as borontrifluoride, aluminumtrichloride, ferric (III) chloride, tin (IV) chloride, titane (IV) chlorid and zinc (II) chloride, and also organic acids, such as formic acid, acetic acid, propionic acid, oxalic acid, toluene sulfonic acid, benzene sulfonic acid, campher sulfonic acid, citric acid, and trifluoro acetic acid.

In general, the acid is employed in catalytic amounts. It may however also be employed in equimolar amounts, in excess, or as a solvent.

The starting materials are generally reacted with one another in equimolar amounts. In terms of yield, it may be advantageous to use an excess of compounds VII based on compounds V.

Compounds of formula VII can be prepared according to methods known from the literature [e.g. EP-A 89 011 and references cited therein].

Preferably, compounds of formula Id can be prepared by reacting hydrazines of formula V with cyanoalkenes of formula VII wherein L″ is NH₂.

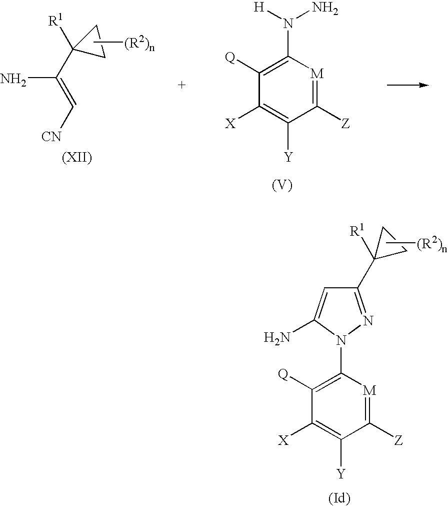

Compounds of formula Ie wherein A is hydrogen, B is hydroxy and the further variables and the index are as defined for formula I can also be prepared by reacting hydrazines of formula V with 3-keto-carboxylic esters of formula VIII wherein the variables and the index are as defined for formula I and R′ is alkyl [Lit.: J. Org. Chem. 1993, 58, 6155-6157].

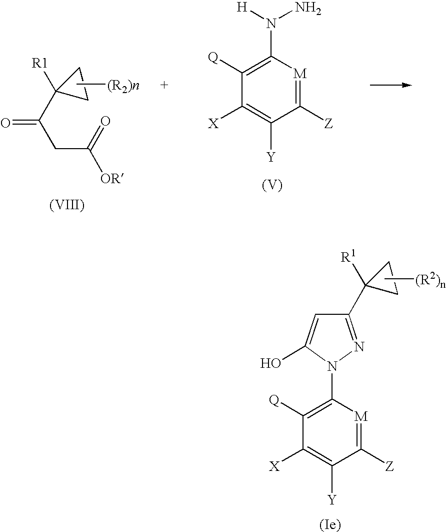

3-Keto-carboxylic acids VIII can be prepared according to the conditions described in the literature [li.: J. Org. Chem. 1978, 43, 2087-2088].

Compounds of formula I wherein A′ is chloro, bromo, nitro, rhodano, or alkylsulfenyl and B is amino can be prepared under conditions described in WO 97/07114 and in the references cited therein by reaction of compounds Id with an electrophile A′-L″ wherein L″ is an electron withdrawing leaving group, such as halogen, e.g. chloro or bromo, or arylsulfonyloxy.

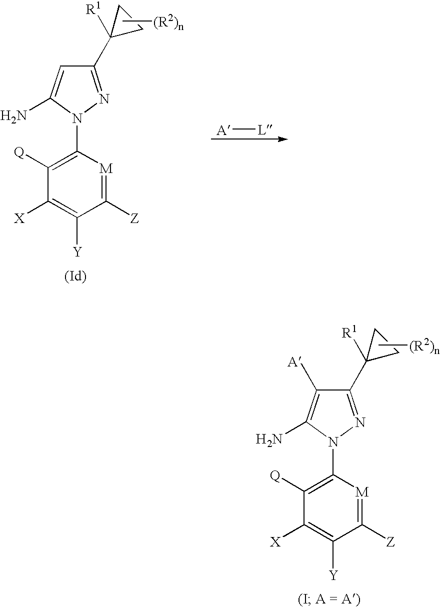

Furthermore, compounds of formula I wherein B is hydroxy, alkoxy, alkoxycarbonylalkyoxy, alkylthio, alkylsulfinyl, or alkylsulfonyl, are obtainable by derivatization of compounds of formula I.

Compounds of formula I wherein B is hydroxy or alkoxy can be prepared by reacting compounds of formula I wherein B is halogen with alkali metal or earth alkali metal alkoxides or alkali metal alcoholates in alcoholes under generally known conditions [lit. WO 97/07114].

Compounds of formula I wherein B is optionally substituted alkoxy can be prepared by reacting compounds of formula I wherein B is hydroxy with optionally substituted alkylhalides under generally known conditions [lit. EP-A 249 033].

Compounds of formula I wherein B is alkylthio can be prepared by reacting compounds of formula I wherein B is amino with dialkyldisulfides under generally known conditions [lit.: J. Chem. Soc. Chem. Commun. 1980, 756-757.].

Compounds of formula I wherein B is alkylsulfinyl can be prepared by reacting compounds of formula I wherein B is alkyl thio with hydrogen peroxide or organic peracids under generally known conditions [lit. Houben-Weyl, “Methoden der organischen Chemie”, IV. Auflage, Bd. 9, pp. 211, Georg Thieme Verlag Stuttgart 1998].

Compounds of formula I wherein B is alkylsulfonyl can be prepared by reacting compounds of formula I wherein B is alkylsulfinyl with hydrogen peroxide or organic peracids under generally known conditions [compare above cited lit., pp. 223].

If individual compounds I are not obtainable by the routes described above, they can be prepared by derivatization of other compounds I.

The reaction mixtures are worked up in a customary manner, for example by mixing with water, phase separation and, if appropriate, chromatographic purification of the crude products. In some cases, the intermediates and end products are obtained in the form of colorless or pale brown viscous oils, which are purified or freed from volatile components under reduced pressure and at moderately elevated temperature. If the intermediates and end products are obtained as solids, they can also be purified by recrystallization or digestion.

The preparation of the pyrazoles of formula I may lead to isomeric mixtures. if desired, however, these can be resolved by the methods customary for this purpose, such as crystallization or chromatography, also on an optically active adsorbate, to give the pure isomers. Pure optically active isomers can be synthesized advantageously from the corresponding optically active starting material.

The 3-substituted-pyrazole compounds of the present invention are effective insect and acarid control agents. Animal pests controlled by the formula I compounds of this invention include for example

insects from the order of the lepidopterans (Lepidoptera), for example Agrotis ypsilon, Agrotis segetum, Alabama argillacea, Anticarsia gemmatalis, Argyresthia conjugella, Autographa gamma, Bupalus piniarius, Cacoecia murinana, Capua reticulana, Cheimatobia brumata, Choristoneura fumiferana, Choristoneura occidentalis, Cirphis unipuncta, Cydia pomonella, Dendrolimus pini, Diaphania nitidalis, Diatraea grandiosella, Earias insulana, Elasmopalpus lignosellus, Eupoecilia ambiguella, Evetria bouliana, Feltia subterranea, Galleria mellonella, Grapholitha funebrana, Grapholitha molesta, Heliothis armigera, Heliothis virescens, Heliothis zea, Hellula undalis, Hibernia defoliaria, Hyphantria cunea, Hyponomeuta malinellus, Keiferia lycopersicella, Lambdina fiscellaria, Laphygma exigua, Leucoptera coffeella, Leucoptera scitella, Lithocolletis blancardella, Lobesia botrana, Loxostege sticticalis, Lymantria dispar, Lymantria monacha, Lyonetia clerkella, Malacosoma neustria, Mamestra brassicae, Orgyia pseudotsugata, Ostrinia nubilalis, Panolis flammea, Pectinophora gossypiella, Peridroma saucia, Phalera bucephala, Phthorimaea operculella, Phyllocnistis citrella, Pieris brassicae, Plathypena scabra, Plutella xylostella, Pseudoplusia includens, Rhyacionia frustrana, Scrobipalpula absoluta, Sitotroga cerealella, Sparganothis pilleriana, Spodoptera frugiperda, Spodoptera littoralis, Spodoptera litura, Thaumatopoea pityocampa, Tortrix viridana, Trichoplusia ni and zeiraphera canadensis,

beetles (Coleoptera), for example Agrilus sinuatus, Agriotes lineatus, Agriotes obscurus, Amphimallus solstitialis, Anisandrus dispar, Anthonomus grandis, Anthonomus pomorum, Atomaria linearis, Blastophagus piniperda, Blitophaga undata, Bruchus rufimanus, Bruchus pisorum, Bruchus lentis, Byctiscus betulae, Cassida nebulosa, Cerotoma trifurcata, Ceuthorrhynchus assimilis, Ceuthorrhynchus napi, Chaetocnema tibialis, Conoderus vespertinus, Crioceris asparagi, Diabrotica longicornis, Diabrotica 12-punctata, Diabrotica virgifera, Epilachna varivestis, Epitrix hirtipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus communis, Meligethes aeneus, Melolontha hippocastani, Melolontha melolontha, Oulema oryzae, Ortiorrhynchus sulcatus, Otiorrhynchus ovatus, Phaedon cochleariae, Phyllotreta chrysocephala, Phyllophaga sp., Phyllopertha horticola, Phyllotreta nemorum, Phyllotreta striolata, Popillia japonica, Sitona lineatus and Sitophilus granaria,

dipterans (Diptera), for example Aedes aegypti, Aedes vexans, Anastrepha ludens, Anopheles maculipennis, Ceratitis capitata, Chrysomya bezziana, Chrysomya hominivorax, Chrysomya macellaria, Contarinia sorghicola, Cordylobia anthropophaga, Culex pipiens, Dacus cucurbitae, Dacus oleae, Dasineura brassicae, Fannia canicularis, Gasterophilus intestinalis, Glossina morsitans, Haematobia irritans, Haplodiplosis equestris, Hylemya platura, Hypoderma lineata, Liriomyza sativae, Liriomyza trifolii, Lucilia caprina, Lucilia cuprina, Lucilia sericata, Lycoria pectoralis, Mayetiola destructor, Musca domestica, Muscina stabulans, Oestrus ovis, Oscinella frit, Pegomya hysocyami, Phorbia antiqua, Phorbia brassicae, Phorbia coarctata, Rhagoletis cerasi, Rhagoletis pomonella, Tabanus bovinus, Tipula oleracea and Tipula paludosa,

thrips (Thysanoptera), e.g. Frankliniella fusca, Frankliniella occidentalis, Frankliniella tritici, Scirtothrips citri, Thrips oryzae, Thrips palmi and Thrips tabaci,

hymenopterans (Hymenoptera), e.g. Athalia rosae, Atta cephalotes, Atta sexdens, Atta texana, Hoplocampa minuta, Hoplocampa testudinea, Monomorium pharaonis, Solenopsis geminata and Solenopsis invicta,

heteropterans (Heteroptera), e.g. Acrosternum hilare, Blissus leucopterus, Cyrtopeltis notatus, Dysdercus cingulatus, Dysdercus intermedius, Eurygaster integriceps, Euschistus impictiventris, Leptoglossus phyllopus, Lygus lineolaris, Lygus pratensis, Nezara viridula, Piesma quadrata, Solubea insularis and Thyanta perditor,

homopterans (Homoptera), e.g. Acyrthosiphon onobrychis, Adelges laricis, Aphidula nasturtii, Aphis fabae, Aphis gossypii, Aphis pomi, Aphis sambuci, Brachycaudus cardui, Brevicoryne brassicae, Cerosipha gossypii, Dreyfusia nordmannianae, Dreyfusia piceae, Dysaphis radicola, Dysaulacorthum pseudosolani, Empoasca fabae, Macrosiphum avenae, Macrosiphum euphorbiae, Macrosiphon rosae, Megoura viciae, Metopolophium dirhodum, Myzodes persicae, Myzus cerasi, Nilaparvata lugens, Pemphigus bursarius, Perkinsiella saccharicida, Phorodon humuli, Psylla mali, Psylla piri, Rhopalomyzus ascalonicus, Rhopalosiphum maidis, Sappaphis mala, Sappaphis mali, Schizaphis graminum, Schizoneura lanuginosa, Trialeurodes vaporariorum and Viteus vitifolii,

termites (Isoptera), e.g. Calotermes flavicollis, Leucotermes flavipes, Reticulitermes flavipes, Reticulitermes lucifugus und Termes natalensis,

orthopterans (Orthoptera), e.g. Acheta domestica, Blatta orientalis, Blattella germanica, Forficula auricularia, Gryllotalpa gryllotalpa, Locusta migratoria, Melanoplus bivittatus, Melanoplus femur-rubrum, Melanoplus mexicanus, Melanoplus sanguinipes, Melanoplus spretus, Nomadacris septemfasciata, Periplaneta americana, Schistocerca americana, Schistocerca peregrina, Stauronotus maroccanus and Tachycines asynamorus,

Arachnoidea, such as arachnids (Acarina), e.g. Amblyomma americanum, Amblyomma variegatum, Argas persicus, Boophilus annulatus, Boophilus decoloratus, Boophilus microplus, Brevipalpus phoenicis, Bryobia praetiosa, Dermacentor silvarum, Eotetranychus carpini, Eriophyes sheldoni, Hyalomma truncatum, Ixodes ricinus, Ixodes rubicundus, Ornithodorus moubata, Otobius megnini, Paratetranychus pilosus, Dermanyssus gallinae, Phyllocoptruta oleivora, Polyphagotarsonemus latus, Psoroptes ovis, Rhipicephalus appendiculatus, Rhipicephalus evertsi, Sarcoptes scabiei, Tetranychus cinnabarinus, Tetranychus kanzawai, Tetranychus pacificus, Tetranychus telarius and Tetranychus urticae, and

Siphonatera, e.g. Xenopsylla cheopsis, Ceratophyllus spp.

Advantageously, the compounds of the invention may be used for the control of insects such as termites, aphids or the like; and acarids such as mites, spiders or the like.

For controlling animal pests, pesticidally active amounts of compounds of formula I are typically applied to the pests or to their food supply, habitat or breeding ground. For the protection of growing plants from attack or infestation by the pests, pesticidally active amounts of the compounds of formula I are typically applied to the foliage, stem or roots of the plants or to the soil or water in which they are growing.

Effective amounts suitable for use in the method of invention may vary depending upon the particular formula I compound, target pest, method of application, application timing, weather conditions, insect or acarid habitat, or the like.

The rate of application of active ingredient for controlling animal pests is from 0.01 to 100, preferably 0.1 to 3 kg/ha under field conditions.

The compounds I can be converted into the customary formulations, e.g. an emulsifiable concentrate, a flowable concentrate, a wettable powder, a microemulsion, a dry compacted granule, a water dispersable granule, a dust, a dust concentrate, a suspension concentrate, a solution, a powder, a paste or any conventional form which is suitable for seed, soil, water, foilage, wood or wooden structure application. The use form depends on the particular purpose; in any case, it should guarantee a fine and uniform distribution of the compound according to the invention.

The composition of the invention comprises an inert agronomically acceptable solid or liquid carrier and an insecticidally or acaricidally effective amount of a compound of formula I.

Carriers suitable for use in the composition of the invention include any material with which the active ingredient is formulated to facilitate application to the locus to be treated. The carrier may be a solid or a liquid including one which facilitates the dilution process. Thus, preferably at least one carrier is a surfactant. For example, the composition may contain two or more carriers, at least one of which is a surfactant.

The formulations are prepared in a known manner, e.g. by extending the active ingredient with solvents and/or carriers, if desired using emulsifiers and dispersants, it also being possible to use other organic solvents as auxiliary solvents if water is used as the diluent. Auxiliaries which are suitable are essentially: solvents such as aromatics (e.g. xylene), chlorinated aromatics (e.g. chlorobenzenes), paraffins (e.g. mineral oil fractions), alcohols (e.g. methanol, butanol), ketones (e.g. cyclohexanone), amines (e.g. ethanolamine, dimethylformamide) and water; carriers such as ground natural minerals (e.g. kaolins, clays, talc, chalk) and ground synthetic minerals (e.g. highly-disperse silica, silicates); emulsifiers such as non-ionic and anionic emulsifiers (e.g. polyoxyethylene fatty alcohol ethers, alkylsulfonates and arylsulfonates) and dispersants such as lignin-sulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates and fatty acids and their alkali metal and alkaline earth metal salts, salts of sulfated fatty alcohol glycol ether, condensates of sulfonated naphthalene and naphthalene derivatives with formaldehyde, condensates of naphthalene or of napthalenesulfonic acid with phenol or formaldehyde, polyoxyethylene octylphenyl ether, ethoxylated isooctylphenol, octylphenol, nonylphenol, alkylphenol polyglycol ethers, tributylphenyl polyglycol ethers, alkylaryl polyether alcohols, isotridecyl alcohol, fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignin-sulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, e.g. benzene, toluene, xylene, paraffin, tetrahydronaphthalene, alkylated naphthalenes or their derivatives, methanol, ethanol, propanol, butanol, chloroform, carbon tetrachloride, cyclohexanol, cyclohexanone, chlorobenzene, isophorone, strongly polar solvents, e.g. dimethylformamide, dimethyl sulfoxide, N-methylpyrrolidone and water.

Powders, materials for scattering and dusts can be prepared by mixing or concomitantly grinding the active substances with a solid carrier.

Granules, e.g. coated granules, impregnated granules and homogeneous granules, can be prepared by binding the active ingredients to solid carriers. Examples of solid carriers are mineral earths, such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, e.g. ammonium sulfate, ammonium phosphate, ammonium nitrate, ureas, and products of vegetable origin, such as cereal meal, tree bark meal, wood meal and nutshell meal, cellulose powders and other solid carriers.

In general, the inventive composition may be in a concentrated form for the convenience of the end-user and for ease of transportation and storage.

In general, the formulations comprise from 0.001% to 95% by weight, preferably from 0.1 to 90% by weight of the active ingredient. The doses are usually in the range of about 0.01 to about 0.1%. The active ingredients are employed in a purity of from 90% to 100%, preferably 95% to 100% (according to NMR spectrum).

The following are exemplary formulations:

I. 5 parts by weight of a compound according to the invention are mixed intimately with 95 parts by weight of finely divided kaolin. This gives a dust which comprises 5% by weight of the active ingredient.

II. 30 parts by weight of a compound according to the invention are mixed intimately with a mixture of 92 parts by weight of pulverulent silica gel and 8 parts by weight of paraffin oil which had been sprayed onto the surface of this silica gel. This gives a formulation of the active ingredient with good adhesion properties (comprises 23% by weight of active ingredient).

III. 10 parts by weight of a compound according to the invention are dissolved in a mixture composed of 90 parts by weight of xylene, 6 parts by weight of the adduct of 8 to 10 mol of ethylene oxide and 1 mol of oleic acid N-monoethanolamide, 2 parts by weight of calcium dodecylbenzenesulfonate and 2 parts by weight of the adduct of 40 mol of ethylene oxide and 1 mol of castor oil (comprises 9% by weight of active ingredient).

IV. 20 parts by weight of a compound according to the invention are dissolved in a mixture composed of 60 parts by weight of cyclohexanone, 30 parts by weight of isobutanol, 5 parts by weight of the adduct of 7 mol of ethylene oxide and 1 mol of isooctylphenol and 5 parts by weight of the adduct of 40 mol of ethylene oxide and 1 mol of castor oil (comprises 16% by weight of active ingredient).

V. 80 parts by weight of a compound according to the invention are mixed thoroughly with 3 parts by weight of sodium diisobutylnaphthalene-alpha-sulfonate, 10 parts by weight of the sodium salt of a lignosulfonic acid from a sulfite waste liquor and 7 parts by weight of pulverulent silica gel, and the mixture is ground in a hammer mill (comprises 80% by weight of active ingredient).

VI. 90 parts by weight of a compound according to the invention are mixed with 10 parts by weight of N-methyl-α-pyrrolidone, which gives a solution which is suitable for use in the form of microdrops (comprises 90% by weight of active ingredient).

VII. 20 parts by weight of a compound according to the invention are dissolved in a mixture composed of 40 parts by weight of cyclohexanone, 30 parts by weight of isobutanol, 20 parts by weight of the adduct of 7 mol of ethylene oxide and 1 mol of isooctylphenol and 10 parts by weight of the adduct of 40 mol of ethylene oxide and 1 mol of castor oil. Pouring the solution into 100,000 parts by weight of water and finely distributing it therein gives an aqueous dispersion which comprises 0.02% by weight of the active ingredient.

VIII. 20 parts by weight of a compound according to the invention are mixed thoroughly with 3 parts by weight of sodium diisobutylnaphthalene-α-sulfonate, 17 parts by weight of the sodium salt of a lignosulfonic acid from a sulfite waste liquor and 60 parts by weight of pulverulent silica gel, and the mixture is ground in a hammer mill. Finely distributing the mixture in 20,000 parts by weight of water gives a spray mixture which comprises 0.1% by weight of the active ingredient.

The active ingredients can be used as such, in the form of their formulations or the use forms prepared therefrom, e.g. in the form of directly sprayable solutions, powders, suspensions or dispersions, emulsions, oil dispersions, pastes, dusts, materials for spreading, or granules, by means of spraying, atomizing, dusting, scattering or pouring. The use forms depend entirely on the intended purposes; in any case, this is intended to guarantee the finest possible distribution of the active ingredients according to the invention.

Aqueous use forms can be prepared from emulsion concentrates, pastes or wettable powders (sprayable powders, oil dispersions) by adding water. To prepare emulsions, pastes or oil dispersions, the substances as such or dissolved in an oil or solvent, can be homogenized in water by means of wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active ingredient concentrations in the ready-to-use products can be varied within substantial ranges. In general, they are from 0.0001 to 10%, preferably from 0.01 to 1%.

The active ingredients may also be used successfully in the ultra-low-volume process (ULV), it being possible to apply formulations comprising over 95% by weight of active ingredient, or even the active ingredient without additives.

Various types of oils, herbicides, fungicides, other pesticides, or bactericides may be added to the active ingredients, if appropriate also only immediately prior to use (tank mix). These agents can be admixed with the agents according to the invention in a weight ratio of 1:10 to 10:1.

In the use form as pesticides in crop protection, the compositions according to the invention can also be present together with other active ingredients, e.g. with herbicides, insecticides, growth regulators, fungicides or else with fertilizers. Mixing the compounds I or the compositions comprising them in the use form as pesticides with other pesticides frequently results in a broader pesticidal spectrum of action.

The following list of pesticides together with which the compounds according to the invention can be used, is intended to illustrate the possible combinations, but not to impose any limitation:

Organophosphates: Acephate, Azinphos-methyl, Chlorpyrifos, Chlorfenvinphos, Diazinon, Dichlorvos, Dicrotophos, Dimethoate, Disulfoton, Ethion, Fenitrothion, Fenthion, Isoxathion, Malathion, Methamidophos, Methidathion, Methyl-Parathion, Mevinphos, Monocrotophos, Oxydemeton-methyl, Paraoxon, Parathion, Phenthoate, Phosalone, Phosmet, Phosphamidon, Phorate, Phoxim, Pirimiphos-methyl, Profenofos, Prothiofos, Sulprophos, Triazophos, Trichlorfon;

Carbamates: Alanycarb, Benfuracarb, Carbaryl, Carbosulfan, Fenoxycarb, Furathiocarb, Indoxacarb, Methiocarb, Methomyl, Oxamyl, Pirimicarb, Propoxur, Thiodicarb, Triazamate;

Pyrethroids: Bifenthrin, Cyfluthrin, Cypermethrin, Deltamethrin, Esfenvalerate, Ethofenprox, Fenpropathrin, Fenvalerate, Cyhalothrin, Lambda-Cyhalothrin, Permethrin, Silafluofen, Tau-Fluvalinate, Tefluthrin, Tralomethrin, Zeta-Cypermethrin;

Arthropod growth regulators: a) chitin synthesis inhibitors: benzoylureas: Chlorfluazuron, Diflubenzuron, Flucycloxuron, Flufenoxuron, Hexaflumuron, Lufenuron, Novaluron, Teflubenzuron, Triflumuron; Buprofezin, Diofenolan, Hexythiazox, Etoxazole, Clofentazine; b) ecdysone antagonists: Halofenozide, Methoxyfenozide, Tebufenozide; c) juvenoids: Pyriproxyfen, Methoprene, Fenoxycarb; d) lipid biosynthesis inhibitors: Spirodiclofen;

Various: Abamectin, Acequinocyl, Amitraz, Azadirachtin, Bifenazate, Cartap, Chlorfenapyr, Chlordimeform, Cyromazine, Diafenthiuron, Dinetofuran, Diofenolan, Emamectin, Endosulfan, Fenazaquin, Fipronil, Formetanate, Formetanate, Hydrochloride, Hydramethylnon, Imidacloprid, Indoxacarb, Pyridaben, Pymetrozine; Spinosad, Sulfur, Tebufenpyrad, Thiamethoxam, and Thiocyclam.

This invention also provides a method for treating, curing, controlling, preventing and protecting warm-blooded animals, including humans, and fish against infestation and infection by helminths, acarids and arthropod endo- and ectoparasites which comprises orally, topically or parenterally administering or applying to said animals an anthelmintically, acaricidally or endo- or ectoparasiticidally effective amount of compounds of formula I.

The above method is particularly useful for controlling and preventing helminth, acarid and arthropod endo- and ectoparasitic infestations and infections in warm-blooded animals such as cattle, sheep, swine, camels, deer, horses, poultry, fish, rabbits, goats, mink, fox, chinchillas, rabbits, dogs and cats as well as humans.

Compounds of formula I are especially useful in controlling helminths and nematodes. Examples for helminths are members of the class Trematoda, commonly known as flukes or flatworms, especially members of the genera Fasciola, Fascioloides, Paramphistomum, Dicrocoelium, Eurytrema, Ophisthorchis, Fasciolopsis, Echinostoma and Paragonimus. Nematodes which can be controlled by the formula I compounds include the genera Haemonchus, Ostertagia, Cooperia, Oesphagastomum, Nematodirus, Dictyocaulus, Trichuris, Dirofilaria, Ancyclostoma, Ascaris and the like.

The formula I compounds of this invention also control endoparasitic arthropod infestations such as cattle grub and stomach bot. In addition, acarid and arthropod ectoparasitic infestations in warm-blooded animals and fish including biting lice, sucking lice, bot flies, biting flies, muscoid flies, myiasitic fly larvae, gnats, mosquitoes, fleas, mites, ticks, nasal bots, keds and chiggers may be controlled, prevented or eliminated by the compounds of this invention. Biting lice include members of Mallophaga such as Bovicola bovis, Trichodectes canis and Damilina ovis. Sucking lice include members of Anoplura such as Haematopinus eurysternus, Haematopinus suis, Linognathus vituli and Solenopotes capillatus. Biting flies include members of Haematobia. Ticks include Boophilus, Rhipicephalus, Ixodes, Hyalomma, Amblyomma and Dermacentor. The formula I compounds may also be used to control mites which are parasitic on warm-blooded mammals and poultry including mites of the orders Acariformes and Parasitiformes.

For oral administration to warm-blooded animals, the formula I compounds may be formulated as animal feeds, animal feed premixes, animal feed concentrates, pills, solutions, pastes, suspensions, drenches, gels, tablets, boluses and capsules. In addition, the formula I compounds may be administered to the animals in their drinking water. For oral administration, the dosage form chosen should provide the animal with about 0.01 mg/kg to 100 mg/kg of animal body weight per day of the formula I compound.

Alternatively, the formula I compounds may be administered to animals parenterally, for example, by intraruminal, intramuscular, intravenous or subcutaneous injection. The formula I compounds may be dispersed or dissolved in a physiologically acceptable carrier for subcutaneous injection. Alternatively, the formula I compounds may be formulated into an implant for subcutaneous administration. In addition the formula I compound may be transdermally administered to animals. For parenteral administration, the dosage form chosen should provide the animal with about 0.01 mg/kg to 100 mg/kg of animal body weight per day of the formula I compound.

The formula I compounds may also be applied topically to the animals in the form of dips, dusts, powders, collars, medallions, sprays and pour-on formulations. For topical application, dips and sprays usually contain about 0.5 ppm to 5,000 ppm and preferably about 1 ppm to 3,000 ppm of the formula I compound. In addition, the formula I compounds may be formulated as ear tags for animals, particularly quadrupeds such as cattle and sheep.

The formula I compounds of this invention may also be used in combination or conjunction with one or more other parasiticidal compounds including anthelmintics, such as benzimidazoles, piperazine, levamisole, pyrantel, and praziquantel; endectocides such as avermectins, and milbemycins; ectoparasiticides such as arylpyrroles, organophosphates, and carbamates, gamabutyric acid inhibitors including fipronil, pyrethroids, spinosads and imidacloprid; insect growth regulators such as pyriproxyfen, and cyromazine; and chitin synthase inhibitors such as benzoylureas including flufenoxuron.

The formula I compounds may also be used in combination or conjunction with one or more compounds selected from piperonyl butoxide, N-octyl bicycloheptene dicarboximide, dipropyl pyridine-2,5-dicarboxylate and 1,5a,6,9,9a,9b-hexahydro-4a(4H)dibenzo-furancarboxaldehyde to broaden the spectrum of activity.

The parasiticidal compositions of the present invention include a parasiticidally effective amount of a formula I compound of this invention or combinations thereof admixed with one or more physiologically tolerable inert, solid or liquid carriers known from veterinary medicinal practice for oral, percutaneous and topical administration. Such compositions may comprise further additives, such as stabilizers, anifoams, viscosity regulators, binders and tackifiers. Whereas commercial products will preferably be formulated as concentrates, the end user will normally employ dilute formulations.

### SYNTHESIS EXAMPLES

With due modification of the starting compounds, the protocols shown in the synthesis examples below were used for obtaining further compounds I. The resulting compounds, together with physical data, are listed in Table I which follows.

### Example 1

### Preparation of Methyl 1-(2,2-dibromo-1-methylcyclopropyl carboxylate

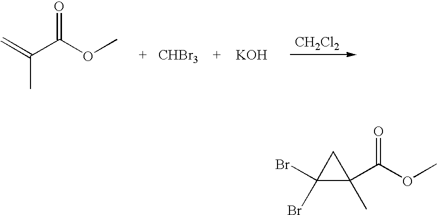

A slurry of powdered KOH (13.2 g of 85%; 0.2 mol) in CH₂Cl₂ was cooled to 0-5° C., treated dropwise with a mixture of CHBr₃ (30.2 g, 0.12 mol) and methyl methacrylate (10 g, 0.1 mol) in CH₂Cl₂ over a 1.5 hr. period, stirred at 0-5° C. for 1 hr, stirred at room temperature for 12 hours, and poured into water. The phases were separated, the organic phase was washed with saturated NaCl, dried with MgSO₄, filtered and evaporated to leave a brown oil. The oil was subjected to Kugelrohr bulb-to-bulb distillation to give 14 g (52% yield) of the title compound as a clear oil of bp. 55°-65° C. at 1.3×10⁻⁴ bar.

### Example 2

### Preparation of 1-(2,2-dibromo-1-methylcyclopropyl) carboxylic acid

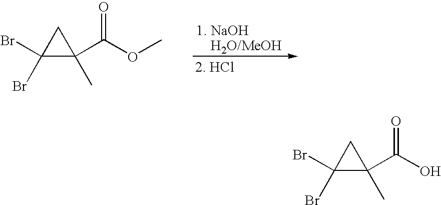

Aqueous 10% NaOH was added to a solution of methyl 2,2-dibromo-1-methylcyclopropane carboxylate (2.71 g, 0.01 mol) in CH₃OH. The reaction mixture was stirred at room temperature for 20 hours, cooled to 5-10° C., acidified with 10% aqueous HCl, stirred for 15 minutes, filtered, washed with water and air dried to give 1.41 g (55% yield) of the title compound (mp. 112-114° C.).

### Example 3

### Preparation of 2,2-dichloro-1-methylcyclopropane carboxylic acid, (2,6-dichloro-4-trifluoromethylphenyl)hydrazide

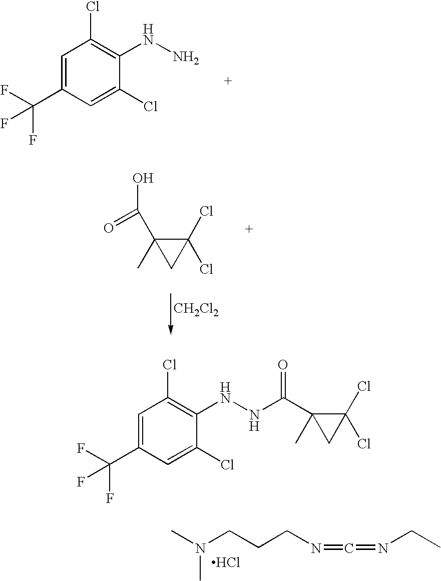

A solution of 2,6-dichloro-4-trifluoromethylphenyl hydrazine (24.5 g, 0.1 mol) and 2,2-dichloro-1-methylcyclopropane-carboxylic acid prepared analogously to examples 1 and 2 (16.9 g, 0.1 mol) in CH₂Cl₂ was treated portionwise with 1-(3-dimethyl-aminopropyl)-3-ethylcarbodiimide hydrochloride (19.2 g, 0.1 mol) over a 15 min. period, stirred at room temperature for 18 hr, quenched with water, stirred for 30 minutes, filtered and air-dried to give 32.3 g (87% yield) of the title compound as an off-white solid (mp. 172-173° C.).

### Example 4

### Preparation of 2,2-Dichloro-1-methylcyclopropane carbonyl chloride, (2, 6-dichloro-4-trifluoromethylphenyl)hydrazone

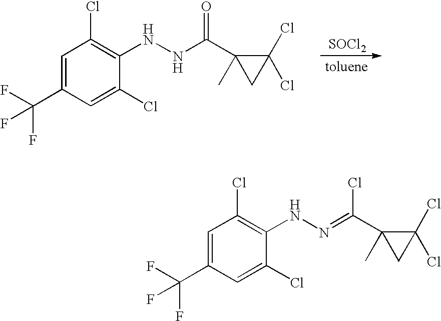

A slurry of the hydrazide of example 3 in toluene was treated with thionyl chloride (31 g, 0.26 mol), heated at reflux temperature for 4 hr., cooled to room temperature, concentrated in vacuo to give a residue, which was dissolved in hexane and filtered through a pad of silica gel. The filtrate was concentrated in vacuo to give 32 g (89% yield) of the product as a pale yellow solid (89% yield; mp. 71-73° C.).

### Example 5

### Preparation of 3-(2,2-dichloro-1-methylcyclopropyl)-1-(2,6-dichloro-4-trifluoromethylphenyl)pyrazole-4-carbonitrile

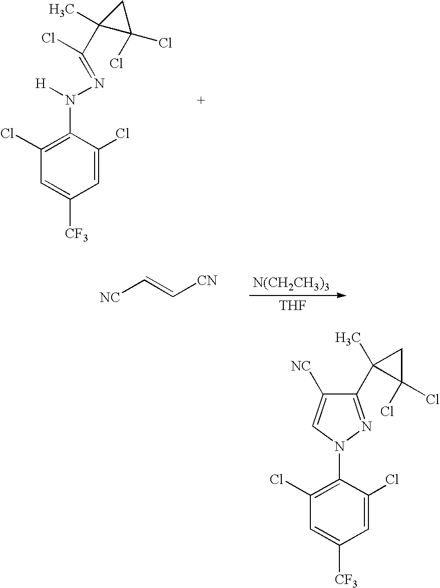

A mixture of the hydrazonyl chloride of example 4 (2.07 g, 0.005 mol) and fumaronitrile (0.47 g, 0.006 mol) in tetrahydrofuran (THF) was treated dropwise with triethylamine (1.01 g, 0.01 mol), stirred at room temperature overnight, quenched with of water, and extracted with ether. The extracts were combined, washed with water and saturated sodium chloride solution, dried over MgSO₄ and concentrated in vacuo to give a brown semi-solid. Chromatography on silica gel and elution with hexanes:ethyl acetate (9:1) gave 0.95 g (44% yield) of the title compound as an off-white solid (mp. 97-98.5° C.).

### Example 6

### Preparation of 5-amino-3-(2,2-Dichloro-1-methylcyclopropyl)-1-(2,6-dichloro-4-trifluoromethylphenyl)pyrazole-4-carbonitrile

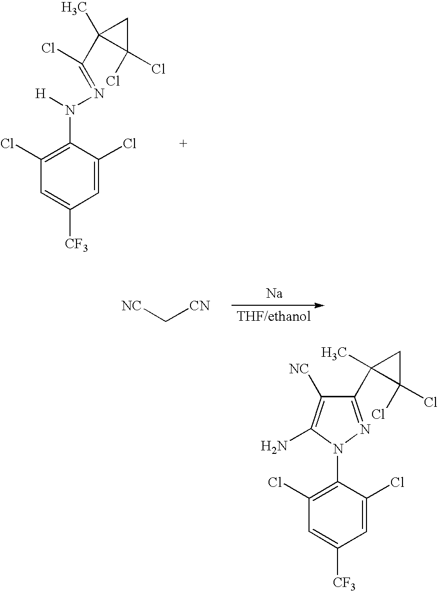

Na metal (2.56 g) was dissolved in 150 ml dry ethanol. The solution was cooled to 0° C. and a solution of the hydrazonyl chloride of example 4 (20.72 g) and 3.48 g malononitrile in 250 ml ethanol/THF (75:25) was added over 2,5 hr. After stirring for an additional 3 hr., the mixture was quenched with water and saturated aqueous NaCl, dried with MgSO₄, filtered and evaporated yielding 22 g of the title compound as yellow crystals (m.p. 209-210° C.).

TABLE I

| | |
|---|---|
| | (I.1) |
| 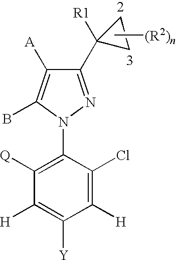 | |

| | | | | | | | | Physical |
|---|---|---|---|---|---|---|---|---|
| | | | | | | | | data: |
| No. | A | B | Q | Y | R¹ | R² | n | m.p. (° C.) |
| I.1-1 | CN | H | Cl | CF₃ | CH₃ | 2,2-Cl₂ | 2 | 97-98.5 |
| I.1-2 | H | CN | Cl | CF₃ | CH₃ | 2,2-Cl₂ | 2 | 110-111 |
| I.1-3 | CN | H | Cl | Cl | CH₃ | 2,2-Cl₂ | 2 | 119-121 |
| I.1-4 | H | CN | Cl | CF₃ | CH₃ | 2,2-Cl₂, | 3 | 123-125 |
| | | | | | | 3-CH₃ | | |
| I.1-5 | CN | Br | Cl | CF₃ | CH₃ | 2,2-Br₂ | 2 | 110-114 |
| I.1-6 | CN | F₃CS | Cl | CF₃ | CH₃ | 2,2-Cl₂ | 2 | — |
| I.1-7 | CN | Br | Cl | H | CH₃ | 2,2-Cl₂ | 2 | 118-120 |
| I.1-8 | CN | CH₃S | Cl | CF₃ | CH₃ | 2,2-Br₂ | 2 | 56-59 |
| I.1-9 | CN | CH₃S | Cl | H | CH₃ | 2,2-Cl₂ | 2 | 118-120 |
| I.1-10 | CN | I | Cl | Cl | CH₃ | 2,2-Cl₂ | 2 | 137-140 |
| I.1-11 | H | CN | Cl | Cl | 4-Cl—C₆H₄ | — | 0 | 125-128 |
| I.1-12 | H | CN | Cl | CF₃ | 2,4-Cl₂—C₆H₃ | — | 0 | 96-98 |
| I.1-13 | H | CN | Cl | CF₃ | CH₃ | 2,2-Cl₂, | 3 | 110-112 |
| | | | | | | 3-CH₃ | | |
| I.1-14 | CN | H | Cl | CF₃ | CH₃ | — | 0 | — |
| I.1-15 | H | CN | Cl | CF₃ | CH₃ | — | 0 | — |
| I.1-16 | H | CN | Cl | CF₃ | 4-Cl—C₆H₄ | — | 0 | — |
| I.1-17 | H | CN | Cl | CF₃ | 4-(CH₃O)—C₆H₄ | — | 0 | — |
| I.1-18 | CN | H | Cl | CF₃ | 4-(CH₃O)—C₆H₄ | — | 0 | — |
| I.1-19 | CN | H | Cl | CF₃ | 4-Cl—C₆H₄ | — | 0 | — |
| I.1-20 | CN | H | Cl | Cl | CH₃ | — | 0 | — |
| I.1-21 | H | CN | Cl | Cl | CH₃ | — | 0 | — |
| I.1-22 | CN | H | Cl | Cl | 4-Cl—C₆H₄ | — | 0 | — |
| I.1-23 | CN | H | Cl | Cl | 4-(CH₃O)—C₆H₄ | — | 0 | — |
| I.1-24 | H | CN | Cl | Cl | 4-(CH₃O)—C₆H₄ | — | 0 | — |
| I.1-25 | CN | H | Cl | CF₃ | CH₃ | 2,2-Br₂ | 2 | — |
| I.1-26 | CN | H | Cl | Cl | CH₃ | 2,2-Br₂ | 2 | — |
| I.1-27 | H | CN | Cl | Cl | CH₃ | 2,2-Br₂ | 2 | — |
| I.1-28 | H | CN | Cl | CF₃ | CH₃ | 2,2-Br₂ | 2 | — |
| I.1-29 | CN | H | Cl | CF₃ | 4-CH₃—C₆H₄ | — | 0 | — |
| I.1-30 | H | CN | Cl | CF₃ | 4-CH₃—C₆H₄ | — | 0 | — |
| I.1-31 | CN | H | Cl | CF₃ | 2,4-Cl₂—C₆H₃ | — | 0 | — |
| I.1-32 | CN | H | Cl | Cl | 2,4-Cl₂—C₆H₃ | — | 0 | — |
| I.1-33 | CN | NH₂ | Cl | CF₃ | CH₃ | 2,2-Cl₂ | 2 | — |
| I.1-34 | CN | Cl | Cl | CF₃ | CH₃ | 2,2-Cl₂ | 2 | — |
| I.1-35 | CN | Cl | Cl | CF₃ | CH₃ | 2,2-Br₂ | 2 | 95-98 |
| I.1-36 | CN | NH₂ | Cl | CF₃ | CH₃ | 2,2-Br₂ | 2 | — |
| I.1-37 | CN | NH₂ | Cl | Cl | CH₃ | 2,2-Cl₂ | 2 | 185-190 |
| I.1-38 | CN | Cl | Cl | Cl | CH₃ | 2,2-Cl₂ | 2 | 128-132 |
| I.1-39 | CN | Br | Cl | Cl | CH₃ | 2,2-Cl₂ | 2 | 133-134 |
| I.1-40 | CN | Br | Cl | CF₃ | CH₃ | 2,2-Cl₂ | 2 | 123-124 |
| I.1-41 | CN | NO₂ | Cl | CF₃ | CH₃ | 2,2-Cl₂ | 2 | — |
| I.1-42 | CN | I | Cl | CF₃ | CH₃ | 2,2-Cl₂ | 2 | 128-130 |
| I.1-43 | CN | CH₃OCH═N | Cl | CF₃ | CH₃ | 2,2-Cl₂ | 2 | 89-91 |
| I.1-44 | CN | (CH₃)₂N | Cl | CF₃ | CH₃ | 2,2-Cl₂ | 2 | 114-115 |
| I.1-45 | CN | (C₂H₅)₂N | Cl | CF₃ | CH₃ | 2,2-Cl₂ | 2 | 122-123 |
| I.1-46 | CN | C₂H₅OCH═N | Cl | CF₃ | CH₃ | 2,2-Cl₂ | 2 | 82-84 |
| I.1-47 | CN | n-C₃H₇OCH═N | Cl | CF₃ | CH₃ | 2,2-Cl₂ | 2 | — |
| I.1-48 | CN | NH₂ | Cl | H | CH₃ | 2,2-Cl₂ | 2 | 225-226 |
| I.1-49 | CN | Br | F | CF₃ | CH₃ | 2,2-Br₂ | 2 | — |
| I.1-50 | CN | Br | Cl | CF₃ | CH₃ | 2-Br | 1 | — |
| I.1-51 | CN | CH₃O | Cl | CF₃ | CH₃ | 2,2-Cl₂ | 2 | — |
| I.1-52 | CN | CH₃S | Cl | CF₃ | CH₃ | 2,2-Cl₂ | 2 | — |
| I.1-53 | CN | CHF₂O | Cl | CF₃ | CH₃ | 2,2-Cl₂ | 2 | — |
| I.1-54 | CN | CH₃O | Cl | CF₃ | CH₃ | 2,2-Br₂ | 2 | — |
| I.1-55 | CN | H | Cl | CF₃ | CH₃ | 2-Br | 1 | — |
| I.1-56 | CN | OH | Cl | H | CH₃ | 2,2-Cl₂ | 2 | 210-212 |
| I.1-57 | CN | [(CH₃)₂NC(O)]NH | Cl | CF₃ | CH₃ | 2,2-Cl₂ | 2 | 67-68 |
| I.1-58 | CN | [C₂H₅OC(O)]₂N | Cl | CF₃ | CH₃ | 2,2-Cl₂ | 2 | — |
| I.1-59 | CN | CH₂═C[CH₃OC(O)]CH₂NH | Cl | CF₃ | CH₃ | 2,2-Cl₂ | 2 | — |
| I.1-60 | CN | CH₃S(O) | Cl | CF₃ | CH₃ | 2,2-Br₂ | 2 | 76-79 |
| I.1-61 | CN | CH₃S(O)₂ | Cl | CF₃ | CH₃ | 2,2-Br₂ | 2 | 70-71 |
| I.1-62 | CN | Br | Cl | CF₃ | C₂H₅OCH₂ | 2,2-Cl₂ | 2 | — |
| I.1-63 | CN | Br | Cl | CF₃ | Cl₂HC═CH | 2,2-Cl₂ | 2 | — |
| I.1-64 | CN | NH₂ | (CH₃)₂N | CF₃ | CH₃ | 2,2-Br₂ | 2 | 98-100 |
| I.1-65 | CN | (CH₃)₂NCH═N | Cl | CF₃ | CH₃ | 2,2-Cl₂ | 2 | 133-134 |
| I.1-66 | CN | [C₂H₅OC(O)]NH | Cl | CF₃ | CH₃ | 2,2-Cl₂ | 2 | — |
| I.1-67 | CN | NH₂ | Cl | CF₃ | Cl₂HC═CH | 2,2-Cl₂ | 2 | — |
| I.1-68 | CN | [(CH₃)₃CC(O)]NH | Cl | CF₃ | CH₃ | 2,2-Cl₂ | 2 | — |
| I.1-69 | CN | [CH₃OC(O)]CH₂NH | Cl | CF₃ | CH₃ | 2,2-Cl₂ | 2 | — |
| I.1-70 | CN | {CH₂═C[CH₃OC(O)]CH₂}₂N | Cl | CF₃ | CH₃ | 2,2-Cl₂ | 2 | — |
| I.1-71 | CN | NH₂ | Cl | CF₃ | C₂H₅OCH₂ | 2,2-Cl₂ | 2 | — |
| I.1-72 | CN | OH | Cl | CF₃ | CH₃ | 2,2-Br₂ | 2 | 88-92 |
| I.1-73 | CN | Br | (CH₃)₂N | CF₃ | CH₃ | 2,2-Br₂ | 2 | 68-71 |
| I.1-74 | CN | Br | CH₃O | CF₃ | CH₃ | 2,2-Cl₂ | 2 | 60-66 |
| I.1-75 | CN | OH | Cl | CF₃ | CH₃ | 2,2-Cl₂ | 2 | 178-180 |
| I.1-76 | CN | [C₂H₅OC(O)]CH₂S | Cl | CF₃ | CH₃ | 2,2-Cl₂ | 2 | — |
| I.1-77 | CN | [(CH₃)₂NSO]₂N | Cl | CF₃ | CH₃ | 2,2-Cl₂ | 2 | 102-104 |
| I.1-78 | CN | CH₃O | CH₂═CHCH₂O | CF₃ | CH₃ | 2,2-Cl₂ | 2 | — |
| I.1-79 | CN | I | CH₃O | Cl | CH₃ | 2,2-Cl₂ | 2 | 75-78 |
| I.1-80 | CN | CH₂═CHCH₂O | CH₂═CHCH₂O | CF₃ | CH₃ | 2,2-Cl₂ | 2 | — |
| I.1-81 | CN | CH₃S(O) | Cl | CF₃ | CH₃ | 2,2-Cl₂ | 2 | — |
| I.1-82 | CN | CH₃S(O) | Cl | H | CH₃ | 2,2-Cl₂ | 2 | 128-130 |
| I.1-83 | CN | CH₃S(O) | Cl | H | CH₃ | 2,2-Cl₂ | 2 | 128-130 |
| I.1-84 | CN | NH₂ | (CH₃)₂N | CF₃ | CH₃ | 2,2-Cl₂ | 2 | 88-90 |
| I.1-85 | CN | Br | (CH₃)₂N | CF₃ | CH₃ | 2,2-Cl₂ | 2 | 58-60 |
| I.1-86 | CN | OH | CH₂═CHCH₂O | CF₃ | CH₃ | 2,2-Cl₂ | 2 | — |
| I.1-87 | CN | n-C₃H₇O | Cl | CF₃ | CH₃ | 2,2-Cl₂ | 2 | 83-84 |
| I.1-88 | CN | Br | CH₃O | CF₃ | CH₃ | 2,2-Br₂ | 2 | — |
| I.1-89 | CN | H | CH₃O | CF₃ | CH₃ | 2,2-Br₂ | 2 | — |
| I.1-90 | CN | [CH₃OC(O)]CH₂O | Cl | CF₃ | CH₃ | 2,2-Cl₂ | 2 | — |
| I.1-91 | CN | Br | CF₃CH₂O | CF₃ | CH₃ | 2,2-Br₂ | 2 | — |
| I.1-92 | CN | H | CF₃CH₂O | CF₃ | CH₃ | 2,2-Br₂ | 2 | — |
| I.1-93 | CN | [(cyclo-C₃H₇)(O)C]₂N | Cl | CF₃ | CH₃ | 2,2-Cl₂ | 2 | 162-164 |
| I.1-94 | CN | (cyclo-C₃H₇)(O)CNH | Cl | CF₃ | CH₃ | 2,2-Cl₂ | 2 | — |
| I.1-95 | CN | NCCH═CH | Cl | CF₃ | CH₃ | 2,2-Br₂ | 2 | 168-170 |
| I.1-96 | CN | NC(Cl)HCCH₂ | Cl | CF₃ | CH₃ | 2,2-Br₂ | 2 | — |
| I.1-97 | C₂H₅O(O)C | OH | Cl | CF₃ | CH₃ | 2,2-Cl₂ | 2 | 232-235 |
| I.1-98 | H₂N(O)C | Br | Cl | CF₃ | CH₃ | 2,2-Br₂ | 2 | 183-185 |
| I.1-99 | HO(O)C | H | Cl | CF₃ | CH₃ | 2,2-Cl₂ | 2 | 192-194 |
| I.1-100 | C₂H₅O(O)C | NH₂ | Cl | CF₃ | CH₃ | 2,2-Cl₂ | 2 | 165-180 |
| I.1-101 | C₂H₅O(O)C | Cl | Cl | CF₃ | CH₃ | 2,2-Cl₂ | 2 | 156-160 |
| I.1-102 | CN | {[H₃CO(O)C]C═CH—[C(O)OCH₃]}N | Cl | CF₃ | CH₃ | 2,2-Cl₂ | 2 | — |
| I.1-103 | H₃CO(O)C | NH₂ | Cl | CF₃ | CH₃ | 2,2-Cl₂ | 2 | — |
| I.1-104 | H₃CO(O)C | Br | Cl | CF₃ | CH₃ | 2,2-Cl₂ | 2 | 141-142 |
| I.1-105 | H₃CO(O)C | H | Cl | CF₃ | CH₃ | 2,2-Cl₂ | 2 | 130-132 |
| I.1-106 | H | CNCH₂O | Cl | CF₃ | CH₃ | 2,2-Cl₂ | 2 | — |
| I.1-107 | H₃CO(O)C | [(H₃CO(O)C(CH₂═)C]CH₂ | Cl | CF₃ | CH₃ | 2,2-Cl₂ | 2 | — |
| I.1-108 | CN | (H₃C)₂CHO(S)CS | Cl | CF₃ | CH₃ | 2,2-Cl₂ | 2 | — |
| I.1-109 | H₃CO(O)C | NH₂ | N(CH₃)₂ | CF₃ | CH₃ | 2,2-Cl₂ | 2 | 75-78 |
| I.1-110 | H₃CO(O)C | Br | N(CH₃)₂ | CF₃ | CH₃ | 2,2-Cl₂ | 2 | — |
| I.1-111 | CN | CNCH═CH | Cl | CF₃ | CH₃ | 2,2-Br₂ | 2 | — |
| I.1-112 | H₂N(O)C | Br | Cl | CF₃ | CH₃ | 2,2-Cl₂ | 2 | 185-186 |
| I.1-113 | CN | NH₂ | C₆H₅(CH₂)—S(CH₂)₃S | CF₃ | CH₃ | 2,2-Cl₂ | 2 | — |
| I.1-114 | CN | NH₂ | Cl | CF₃ | H | 2,2-Cl₂ | 2 | 148-152 |
| I.1-115 | CN | NH₂ | Cl | CF₃ | H | 2,2-Br₂ | 2 | 180-184 |
| I.1-116 | CN | Cl | Cl | CF₃ | H | 2,2-Cl₂ | 2 | * |
| I.1-117 | CN | Br | Cl | CF₃ | H | 2,2-Cl₂ | 2 | ** |

*¹H-NMR[CDCl₃]: δ in ppm: 2.10(dd), 2.32(t), 3.0(dd), 7.8(s).
**¹H-NMR[CDCl₃]: δ in ppm: 2.12(dd), 2.33(t), 3.03(dd), 7.79(s).

Examples of Action Against Animal Pest

The action of the compounds of the formula I against pests was demonstrated by the following experiments:

The active compounds were formulated

a. for testing the activity against aphis gossypii, tetranychus urticae, myzus persicae, and aphis fabae, as 50:50 acetone:water solutions amended with 100 ppm Kinetic® (surfactant),

b. for testing the activity against spodoptera eridania and diabrotica virgifera virgifera Leconte as a 10.000 ppm solution in a mixture of 35% acetone and water, which was diluted with water, if needed,

c. for testing the activity against nilaparvata lugens and sogatella furcifera as a 20:80 acetone:water solution. Surfactant (Alkamuls EL 620) was added at the rate of 0.1% (vol/vol).

After the experiments were completed, in each case the lowest concentration was determined at which the compound still caused an 75 to 100% inhibition or mortality in comparison with untreated controls (limit or minimal concentration).

Cotton Aphid (Aphis gossypii)

Cotton plants in the cotyledon stage (variety ‘Delta Pine’) are infested with approximately 100 laboratory-reared aphids by placing infested leaf sections on top of the test plants. The leaf sections are removed after 24 hr. The cotyledons of the intact plants are dipped into gradient solutions of the test compound. Aphid mortality on the treated plants, relative to mortality on check plants, is determined after 5 days.

In this test, compounds I-2.5, I-2.34, I-2.35, I-2.37, I-2.40, I-2.44, I-2.49, I-2.51, I-2.53, I-2.54, I-2.74, and I-2.90 at 300 ppm showed over 75% mortality in comparison with untreated controls.

Twospotted Spider Mite (Tetranychus urticae)

Lima bean plants in the 1^{st} leaf-pair stage (variety ‘Henderson’) are infested with approximately 100 laboratory-reared mites per leaf by placing infested leaf sections on top of the test plants. The leaf sections are removed after 24 hr. The foliage of the intact plants is dipped into gradient solutions of the test compound. Mite mortality is determined after 5 days.

In this test, compounds I-2.3, I-2.4, I-2.5, I-2.25, I-2.26, I-2.34, I-2.35, I-2.40, I-2.41, I-2.43, and I-2.50 at 300 ppm showed over 75% mortality in comparison with untreated controls.

Green Peach Aphid (Myzus persicae)

Pepper plants in the 2^{nd} leaf-pair stage (variety ‘California Wonder’) are infested with approximately 40 laboratory-reared aphids by placing infested leaf sections on top of the test plants. The leaf sections are removed after 24 hr. The leaves of the intact plants are dipped into gradient solutions of the test compound. Aphid mortality on the treated plants, relative to mortality on check plants, is determined after 5 days.

In this test, compounds I-2.1, I-2.5, I-2.8, I-2.34, I-2.35, I-2.38, I-2.39, I-2.40, I-2.41, I-2.42, I-2.44, I-2.46, I-2.49, I-2.50, I-2.51, I-2.52, I-2.53, and I-2.54 at 300 ppm showed a 100% mortality in comparison with untreated controls.

Bean Aphid (Aphis fabae)

Nasturtium plants in the 1^{st} leaf-pair stage (variety ‘Mixed Jewle’) are infested with approximately 25 laboratory-reared aphids by placing infested cut plants on top of the test plants. The cut plants are removed after 24 hr. The foliage and stem of the test plants are dipped into gradient solutions of the test compound. Aphid mortality is determined after 3 days.

In this test, compounds I-2.1, I-2.4, I-2.5, I-2.11, I-2.13, I-2.25, I-2.26, I-2.34, I-2.35, I-2.38, I-2.50, I-2.51, and I-2.74 at 300 ppm showed over 75% mortality in comparison with untreated controls.

Termites (Reticulitérmes flávipes)

Test arenas are prepared by dispensing a thin layer of 1.5% agar into Petri dishes and then spreading a thin layer of pre-treated soil (NJ sandy loam) over the agar. The soil is prepared by treatment with varying concentrates of the test compound. Termite workers (mid-size or larger) are introduced into the test arena and water is added as needed to maintain soil moisture. The test arenas are maintained at about 27° C. on metal trays, covered with blotting paper for shade, and enclosed in plastic bags to reduce moisture loss. Daily assessments of mortality are made for a 7-day period for mortality and dead insects are removed. Each treatment is replicated 3 to 9 times with 10 termites/replicate. Termite mortality is determined after days.

In this test, compounds I-2.1 at 10 ppm showed a 100% mortality after a 7-day period in comparison with untreated controls.

Cockroaches (Blattella germanica)

Test arenas were prepared from plastic sweater boxes measuring 41 cm (length)×28 cm (width)×15 cm (height). An opening (17×29 cm) was cut into the lide of each box and covered with screening for ventilation purposes. The containers were provided with harborage, water and insecticide bait. One to fourteen-day-old German cockroach adult males (20 adults/treat-ment/replication, two replications per treatment) are introduced into the arenas and the mortality is recorded daily for a maximum of 10 days following treatments. Mortality was considered to be reached when no flight response or upright position could be elicited from prodding.

In this test, compounds I-2.1 at 5% active ingredient in the bait showed over 87% mortality after a 2-day period in comparison with untreated controls.

Southern Armyworm (Spodoptera eridania), 2nd Instar Larvae

A Sieva lima bean leaf expanded to 7-8 cm in length is dipped in the test solution with agitation for 3 seconds and allowed to dry in a hood. The leaf is then placed in a 100×10 mm petri dish containing a damp filter paper on the bottom and ten 2nd instar caterpillars. At 5 days, observations are made of mortality, reduced feeding, or any interference with normal molting.

In this test, compounds I-2.1, I-2.2, I-2.3, I-2.5, I-2.8, I-2.25, I-2.26, I-2.27, I-2.28, I-2.34, I-2.51, I-2.52, I-2.54, I-2.55, I-2.60, I-2.61, I-2.73, I-2.80, I-2.81, I-2.85, and I-2.98 at 300 ppm showed over 75% mortality in comparison with untreated controls.

Brown Plant Hopper (nilaparvata lugens)

White-Backed Plant Hopper (sogatella furcifera)

Potted rice plants of 3-4 weeks of age are sprayed with 10 ml of the test solution using air driven hand atomizer (Devillbis atomizer) at 1.7 bar. The treated plants are allowed to dry for about 1 hour and covered with Mylar cages. The plants are inoculated with 10 adults of each species (5 male and 5 females) and kept at 25-27° C. and 50-60% humidity for 3 days. Mortality is assed after 24, 48 and 72 hours after treatment. Dead insects are usually found in the water surface. Each treatment is replicated once.

In this test, compounds I-2.1, I-2.2, I-2.3, I-2.5, I-2.14, I-2.25, I-2.28, I-2.33, I-2.34, I-2.35, I-2.36, I-2.38, I-2.39, I-2.40, I-2.41, I-2.42, I-2.43, I-2.44, I-2.46, I-2.47, I-2.52, I-2.59, I-2.74, I-2.76, I-2.81, I-2.99, and I-2.108 at 500 ppm showed over 75% mortality of nilaparvata lugens in comparison with untreated controls.

In this test, compounds I-2.1, I-2.2, I-2.3, I-2.4, I-2.5, I-2.14, I-2.25, I-2.28, I-2.33, I-2.35, I-2.38, I-2.39, I-2.40, I-2.41, I-2.43, I-2.44, I-2.46, I-2.47, I-2.52, I-2.59, I-2.74, I-2.81, I-2.98, and I-2.99 at 500 ppm showed over 75% mortality of sogatella furcifera in comparison with untreated controls.

What is claimed is:

1. Compounds of formula I 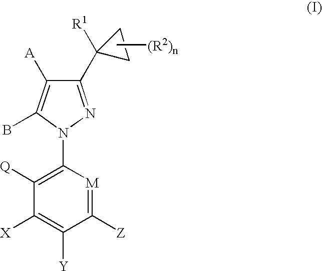
wherein the variables and the index have the following meanings:
R¹ hydrogen, halogen, C₁-C₆-alkyl, C₁-C₆-haloalkyl, C₂-C₆-alkenyl, C₂-C₆-haloalkenyl, C₁-C₆-alkylthio, C₁-C₆-alkoxy-C₁-C₄-alkyl, C₁-C₆-alkylthio-C₁-C₄-alkyl, or phenyl which is unsubstituted or substituted with 1 to 3 groups R^{a};
R^{a} halogen, nitro, cyano, C₁-C₆-alkyl, C₁-C₆-haloalkyl, C₁-C₆-alkylthio, C₁-C₆-haloalkylthio, C₁-C₆-alkoxy or C₁-C₆-haloalkoxy;
R² halogen, C₁-C₆-alkyl, C₁-C₆-haloalkyl, C₂-C₆-alkenyl, C₂-C₆-haloalkenyl or phenyl which is unsubstituted or substituted with 1 to 3 groups R^{a};
A hydrogen, hydroxy, cyano, nitro, halogen, rhodano, C₁-C₆-alkoxy, C₁-C₆-haloalkoxy, C₂-C₆-alkenyloxy, C₁-C₆-alkylthio, C₁-C₆-haloalkylthio, C₁-C₆-alkylsulfinyl, C₁-C₆-alkylsulfonyl, aminothiocarbonyl, hydroxycarbonyl, C₁-C₆-alkoxycarbonyl, aminocarbonyl;
B hydrogen, hydroxy, amino, cyano, nitro, halogen, C₁-C₆-alkyl, unsubstituted or substituted by one to three groups selected from halogen and cyano; C₁-C₆-alkoxy, unsubstituted or substituted by one to three groups selected from halogen, cyano, C₂-C₄-alkenyl, and C₁-C₆-alkoxycarbonyl-C₂-C₄-alkenyl; C₂-C₆-alkenyl, unsubstituted or substituted by one to three groups selected from halogen and cyano; C₂-C₆-alkenyloxy, C₁-C₆-alkylthio, C₁-C₆-haloalkylthio, C₁-C₆-alkoxythiocarbonylthio, C₁-C₆-alkoxycarbonyl-C₁-C₄-alkoxy, C₁-C₆-alkoxycarbonyl-C₁-C₄-alkylthio, C₁-C₆-alkylsulfinyl, C₁-C₆-alkylsulfonyl, aminothiocarbonyl, NR³R⁴, N═CHOR⁵, or N═CHNR⁵;
R³,R⁴ each independently hydrogen, C₁-C₆-alkyl, C₁-C₆-alkoxycarbonyl-C₁-C₄-alkyl, [(C₁-C₆-alkoxycarbonyl)(C₂-C₄-alkenyl)]C₁-C₄-alkyl, C₁-C₆-alkoxycarbonyl-C₂-C₄-alkenyl, C₁-C₆-alkyl-carbonyl, C₃-C₇-cycloalkylcarbonyl, C₁-C₆-alkyl-aminocarbonyl, di-(C₁-C₆-alkyl)aminocarbonyl, C₁-C₆-alkoxycarbonyl, C₁-C₆-alkoxy-aminosulfonyl, or di-(C₁-C₆-alkoxy)aminosulfonyl;
R⁵ C₁-C₆-alkyl, C₁-C₆-haloalkyl, or phenyl-C₁-C₄-alkyl;
Q hydrogen, nitro, halogen, C₁-C₄-haloalkyl, C₁-C₆-alkylamino, di-(C₁-C₆)-alkylamino, C₁-C₆-alkoxy, C₁-C₆-haloalkoxy, C₂-C₆-alkenyloxy;
X hydrogen, halogen, C₁-C₆-haloalkyl, C₁-C₆-alkoxy or C₁-C₆-haloalkoxy;
Y hydrogen, halogen, C₁-C₆-haloalkyl, C₁-C₆-alkoxy or C₁-C₆-haloalkoxy;
Z hydrogen, halogen, C₁-C₆-haloalkyl, C₁-C₆-alkoxy or C₁-C₆-haloalkoxy;
M N or CR⁶;
R⁶ hydrogen, nitro, halogen or C₁-C₄-haloalkyl;
n 0, 1, 2, 3, or 4,
with the proviso that, when R¹ is hydrogen, n is not zero.

2. Compounds of formula I according to claim 1 wherein R¹ is C₁-C₆-alkyl.

3. Compounds of formula I according to claims 1 or 2 wherein R² is halogen.

4. Compounds of formula I according to claims 1 to 3 wherein A is hydrogen, cyano, nitro, or halogen.

5. Compounds of formula I according to claims 1 to 4 wherein B is hydrogen, halogen, C₁-C₆-alkoxy, or C₁-C₆-alkylthio.

6. A method for the control of insects or acarids which comprises contacting said insect or acarid, or their food supply, habitat or breeding grounds with an insecticidally or acaricidally effective amount of a compound of formula I as defined in claims 1 to 5.

7. A method for the protection of a plant from devastation or damage caused by insect or acarid attack or infestation which comprises applying to said plant or the locus in which it is growing or stored an insecticidally or acaricidally effective amount of a compound of formula I as defined in claims 1 to 5.

8. A method for the protection of wood, wood products or wooden structures from infestation and damage caused by wood-eating insects which comprises applying to said wood, wood product or wooden structure an insecticidally effective amount of a compound of formula I as defined in claims 1 to 5.

9. A composition which comprises an agriculturally acceptable solid or liquid carrier and an insecticidally or acaricidally effective amount of a compound of formula I as defined in claims 1 to 5.

10. A process for the preparation of compounds of formula Ib wherein A is cyano, B is amino, and the further variables and the index are as defined for formula I in claims 1 to 3, characterized in that compounds of formula II are reacted with malononitrile in the presence of a base. [chemical structure]
